(19) United States
(12) Patent Application Publication
(10) Pub. No.: US 2023/0170047 A1
(43) Pub. Date: Jun. 1, 2023

(54) GENETIC TESTING METHOD, SIGNATURE EXTRACTION METHOD, APPARATUS, DEVICE, AND SYSTEM

(71) Applicant: Alibaba Singapore Holding Private Limited, Singapore (SG)

(72) Inventors: Han Yang, Hangzhou (CN); Fei GU, Hangzhou (CN)

(21) Appl. No.: 17/832,503

(22) Filed: Jun. 3, 2022

(30) Foreign Application Priority Data
Jun. 10, 2021 (CN) 202110648180.4

(51) Int. Cl. G16B 30/00; G16B 40/20; G16B 20/20; G16H 50/30

(52) U.S. Cl. CPC G16B 30/00; G16B 40/20; G16B 20/20; G16H 50/30

(57) ABSTRACT

A genetic testing method including obtaining a to-be-processed genetic sequence, where an average number of gene fragments corresponding to each position in the genetic sequence is less than or equal to a preset threshold; performing signature extraction on the genetic sequence, and obtaining a gene signature; enhancing the gene signature, and obtaining an enhanced signature corresponding to the gene signature; and testing the genetic sequence based on the enhanced signature, and obtaining a testing result. Based on the technical solutions, signature extraction is performed on the genetic sequence, and the gene signature is obtained; the gene signature is then enhanced, and the enhanced signature is obtained; and then the genetic sequence is tested based on the enhanced signature, and the testing result is obtained. In this way, not only is genetic testing precision ensured, but also data processing costs and the quantity of processed data are further effectively reduced.

### CROSS REFERENCE TO RELATED APPLICATIONS

This application claims priority to Chinese patent application No. 202110648180.4 filed on 10 Jun. 2021 and entitled “GENETIC TESTING METHOD, SIGNATURE EXTRACTION METHOD, APPARATUS, DEVICE, AND SYSTEM,” which is incorporated herein by reference in its entirety.

### TECHNICAL FIELD

The present disclosure relates to the field of genetic testing technologies, and, more particular, to genetic testing methods, signature extraction methods, apparatuses, devices, and systems.

### BACKGROUND

Genetic sequencing is a novel gene testing technology, and can be used to analyze and determine a complete genetic sequence from blood or saliva to predict a possibility of contracting a plurality of diseases, individual behavior characteristics, and behavior rationality. The genetic sequencing technology can be used to lock individual diseased genes, so that prevention and treatment may be carried out in advance based on the individual diseased genes.

A genetic sequence includes a large quantity of reads fragments. The reads fragment is a DNA fragment with a specific length. The foregoing specific length depends on a read length of a sequencer. Information in each reads fragment may include: a base sequence, a quality sequence, positive and negative chains, and the like. The foregoing base sequence is in a one-to-one correspondence with the quality sequence. For humans, a reads fragment covers 23 pairs of chromosomes, totaling more than 3 billion base pairs.

Generally, for humans, it costs thousands of dollars to do a complete genome sequencing. With continuous development of sequencing technologies in recent years, costs of genetic sequencing have been reduced to a certain extent. However, genetic sequencing still costs a lot. Therefore, how to reduce genetic testing costs is an urgent problem to be resolved.

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter. The term “technique(s) or technical solution(s)” for instance, may refer to apparatus(s), system(s), method(s) and/or computer-readable instructions as permitted by the context above and throughout the present disclosure.

Embodiments of the present disclosure provide a genetic testing method, a signature extraction method, an apparatus, a device, and a system. Signature extraction is performed on a low-depth genetic sequence, and a low-depth gene signature is obtained; the gene signature is then enhanced, and testing is performed based on an enhanced signature, thereby not only ensuring genetic testing precision, but also further effectively reducing data processing costs and a quantity of processed data.

According to an example embodiment, the present disclosure provides a genetic testing method, including:

obtaining a to-be-processed genetic sequence, where an average number of gene fragments corresponding to each position in the genetic sequence is less than or equal to a preset threshold;

performing signature extraction on the genetic sequence, and obtaining a gene signature;

enhancing the gene signature, and obtaining an enhanced signature corresponding to the gene signature; and

testing the genetic sequence based on the enhanced signature, and obtaining a testing result.

According to an example embodiment, the present disclosure provides a genetic testing apparatus, including:

a first obtaining module, configured to obtain a to-be-processed genetic sequence, where an average number of gene fragments corresponding to each position in the genetic sequence is less than or equal to a preset threshold;

a first extraction module, configured to perform signature extraction on the genetic sequence, and obtain a gene signature;

a first processing module, configured to enhance the gene signature, and obtain an enhanced signature corresponding to the gene signature; and

a first testing module, configured to test the genetic sequence based on the enhanced signature, and obtain a testing result.

According to an example embodiment, the present disclosure provides an electronic device, including one or more memories and processors, where the memories are configured to store one or more computer instructions, and when the one or more computer instructions are executed by the processors, the above genetic testing method is implemented.

According to an example embodiment, the present disclosure provides a computer storage medium, configured to store a computer program, where the computer program causes a computer to implement the above genetic testing method during execution.

According to an example embodiment, the present disclosure provides a signature extraction method, including:

obtaining a to-be-processed genetic sequence, where an average number of gene fragments corresponding to each position in the genetic sequence is less than or equal to a preset threshold;

performing signature extraction on the genetic sequence, and obtaining a gene signature; and

enhancing the gene signature, and obtaining an enhanced signature corresponding to the gene signature, where a quantity of information included in the enhanced signature is greater than a quantity of information included in the gene signature.

According to an example embodiment, the present disclosure provides a signature extraction apparatus, including:

a second obtaining module, configured to obtain a to-be-processed genetic sequence, where an average number of gene fragments corresponding to each position in the genetic sequence is less than or equal to a preset threshold;

a second extraction module, configured to perform signature extraction on the genetic sequence, and obtain a gene signature; and

a second processing module, configured to enhance the gene signature, and obtain an enhanced signature corresponding to the gene signature, where a quantity of information included in the enhanced signature is greater than a quantity of information included in the gene signature.

According to an example embodiment, the present disclosure provides an electronic device, including one or more memories and processors, where the memories are configured to store one or more computer instructions, and when the one or more computer instructions are executed by the processors, the above signature extraction method is implemented.

According to an example embodiment, the present disclosure provides a computer storage medium, configured to store a computer program, where the computer program causes a computer to implement the above signature extraction method during execution.

According to an example embodiment, the present disclosure provides a genetic testing method, including:

determining, in response to a request for invoking genetic testing, a processing resource corresponding to a genetic testing service; and

performing the following steps by using the processing resource: obtaining a to-be-processed genetic sequence, where an average number of gene fragments corresponding to each position in the genetic sequence is less than or equal to a preset threshold; performing signature extraction on the genetic sequence, and obtaining a gene signature; enhancing the gene signature, and obtaining an enhanced signature corresponding to the gene signature; and testing the genetic sequence based on the enhanced signature, and obtaining a testing result.

According to an example embodiment, the present disclosure provides a genetic testing apparatus, including:

a third obtaining module, configured to determine, in response to a request for invoking genetic testing, a processing resource corresponding to a genetic testing service; and

a third processing module, configured to perform the following steps by using the processing resource: obtaining a to-be-processed genetic sequence, where an average number of gene fragments corresponding to each position in the genetic sequence is less than or equal to a preset threshold; performing signature extraction on the genetic sequence, and obtaining a gene signature; enhancing the gene signature, and obtaining an enhanced signature corresponding to the gene signature; and testing the genetic sequence based on the enhanced signature, and obtaining a testing result.

According to an example embodiment, the present disclosure provides an electronic device, including one or more memories and processors, where the memories are configured to store one or more computer instructions, and when the one or more computer instructions are executed by the processor, the above genetic testing method is implemented.

According to an example embodiment, the present disclosure provides a computer storage medium, configured to store a computer program, where the computer program causes a computer to implement the above genetic testing method during execution.

According to an example embodiment, the present disclosure provides a signature extraction method, including:

determining, in response to a request for invoking signature extraction, a processing resource corresponding to a signature extraction service; and

performing the following steps by using the processing resource: obtaining a to-be-processed genetic sequence, where an average number of gene fragments corresponding to each position in the genetic sequence is less than or equal to a preset threshold; performing signature extraction on the genetic sequence, and obtaining a gene signature; and enhancing the gene signature, and obtaining an enhanced signature corresponding to the gene signature, where a quantity of information included in the enhanced signature is greater than a quantity of information included in the gene signature.

According to an example embodiment, the present disclosure provides a signature extraction apparatus, including:

a fourth obtaining module, configured to determine, in response to a request for invoking signature extraction, a processing resource corresponding to a signature extraction service; and

a fourth processing module, configured to perform the following steps by using the processing resource: obtaining a to-be-processed genetic sequence, where an average number of gene fragments corresponding to each position in the genetic sequence is less than or equal to a preset threshold; performing signature extraction on the genetic sequence, and obtaining a gene signature; and enhancing the gene signature, and obtaining an enhanced signature corresponding to the gene signature, where a quantity of information included in the enhanced signature is greater than a quantity of information included in the gene signature.

According to an example embodiment, the present disclosure provides an electronic device, including one or more memories and processors, where the memories are configured to store one or more computer instructions, and when the one or more computer instructions are executed by the processors, the above signature extraction method is implemented.

According to an example embodiment, the present disclosure provides a computer storage medium, configured to store a computer program, where the computer program causes a computer to implement the above signature extraction method during execution.

According to an example embodiment, the present disclosure provides a genetic testing method, comprising:

performing sample collection on a specified object, and obtaining a to-be-processed sample;

determining a to-be-processed genetic sequence based on the to-be-processed sample, where an average number of gene fragments corresponding to each position in the genetic sequence is less than or equal to a preset threshold;

performing signature extraction on the genetic sequence, and obtaining a gene signature;

enhancing the gene signature, and obtaining an enhanced signature corresponding to the gene signature; and

testing the genetic sequence based on the enhanced signature, and obtaining a testing result.

According to an example embodiment, the present disclosure provides a genetic testing apparatus, comprising:

a fifth collection module, configured to perform sample collection on a specified object, and obtain a to-be-processed sample;

a fifth determining module, configured to determine a to-be-processed genetic sequence based on the to-be-processed sample, where an average number of gene fragments corresponding to each position in the genetic sequence is less than or equal to a preset threshold;

a fifth extraction module, configured to perform signature extraction on the genetic sequence, and obtain a gene signature; and

a fifth processing module, configured to enhance the gene signature, and obtain an enhanced signature corresponding to the gene signature, where the fifth processing module is further configured to test the genetic sequence based on the enhanced signature, and obtain a testing result.

According to an example embodiment, the present disclosure provides an electronic device, including one or more memories and processors, where the memories are configured to store one or more computer instructions, and when the one or more computer instructions are executed by the processors, the above genetic testing method is implemented.

According to an example embodiment, the present disclosure provides a computer storage medium, configured to store a computer program, where the computer program causes a computer to implement the above genetic testing method during execution.

According to an example embodiment, the present disclosure provides a genetic testing system, including:

a genetic sequence collection terminal, configured to obtain a to-be-processed genetic sequence, and transmit the genetic sequence to a genetic testing terminal, where an average number of gene fragments corresponding to each position in the genetic sequence is less than or equal to a preset threshold; and

the genetic testing terminal, in a communication connection with the genetic sequence collection terminal, and configured to obtain the to-be-processed genetic sequence; perform signature extraction on the genetic sequence, and obtain a gene signature; enhance the gene signature, and obtain an enhanced signature corresponding to the gene signature; and test the genetic sequence based on the enhanced signature, and obtain a testing result.

In the technical solution provided by the embodiments of the present disclosure, the to-be-processed genetic sequence is obtained, signature extraction is performed on the genetic sequence, and the gene signature is obtained. The genetic sequence needing to be processed is low-depth gene data. Therefore, the gene signature obtained by performing signature extraction on the low-depth genetic sequence is also a low-depth gene signature, and then the gene signature is enhanced, so that the enhanced signature corresponding to the gene signature is obtained. Then, the genetic sequence is tested based on the enhanced signature, and the testing result is obtained. In this way, not only is genetic testing precision ensured, but also data processing costs and a quantity of processed data are further effectively reduced, thereby effectively achieving relatively precise testing based on the low-depth gene data, further improving practicality of the method, and facilitating promotion and application in the market.

### BRIEF DESCRIPTION OF DRAWINGS

To describe the technical solutions in the example embodiments of the present more clearly, the following briefly describes the accompanying drawings describing the example embodiments of the present disclosure. The accompanying drawings in the following descriptions show some embodiments of the present disclosure, and those of ordinary skill in the art may further derive other accompanying drawings from the accompanying drawings without creative efforts.

FIG. 1 is a schematic diagram of a scenario of a genetic testing method according to an embodiment of the present disclosure;

FIG. 2 is a schematic flowchart of a genetic testing method according to an embodiment of the present disclosure;

FIG. 3 is a schematic flowchart of performing signature extraction on the genetic sequence and obtaining a gene signature according to an embodiment of the present disclosure;

FIG. 4 is a schematic diagram of determining a to-be-analyzed gene fragment corresponding to a genetic sequence according to an embodiment of the present disclosure;

FIG. 5 is a schematic flowchart of a signature extraction method according to an embodiment of the present disclosure;

FIG. 6 is a block diagram of a principle of a genetic testing method according to an application embodiment of the present disclosure;

FIG. 7 is a schematic diagram of a signature converter performing signature extraction according to an application embodiment of the present disclosure;

FIG. 8 is a schematic flowchart of a genetic testing method according to an embodiment of the present disclosure;

FIG. 9 is a schematic flowchart of a signature extraction method according to an embodiment of the present disclosure;

FIG. 10 is a schematic structural diagram of a genetic testing apparatus according to an embodiment of the present disclosure;

FIG. 11 is a schematic structural diagram of an electronic device corresponding to the genetic testing apparatus according to the embodiment shown in FIG. 10;

FIG. 12 is a schematic structural diagram of a signature extraction apparatus according to an embodiment of the present disclosure;

FIG. 13 is a schematic structural diagram of an electronic device corresponding to the signature extraction apparatus according to the embodiment shown in FIG. 12;

FIG. 14 is a schematic structural diagram of another genetic testing apparatus according to an embodiment of the present disclosure;

FIG. 15 is a schematic structural diagram of an electronic device corresponding to the genetic testing apparatus according to the embodiment shown in FIG. 14;

FIG. 16 is a schematic structural diagram of another signature extraction apparatus according to an embodiment of the present disclosure;

FIG. 17 is a schematic structural diagram of an electronic device corresponding to the signature extraction apparatus according to the embodiment shown in FIG. 16;

FIG. 18 is a schematic structural diagram of a genetic testing system according to an embodiment of the present disclosure;

FIG. 19 is a schematic flowchart of another genetic testing method according to an embodiment of the present disclosure;

FIG. 20 is a schematic structural diagram of still another genetic testing apparatus according to an embodiment of the present disclosure; and

FIG. 21 is a schematic structural diagram of an electronic device corresponding to the genetic testing apparatus according to the embodiment shown in FIG. 20.

### DESCRIPTION OF EMBODIMENTS

To make the objectives, technical solutions, and advantages of the embodiments of the present disclosure clearer, the technical solutions in the embodiments of the present disclosure will be described clearly and completely hereinafter in conjunction with the accompanying drawings in the embodiments of the present disclosure. Apparently, the described embodiments are a part of, rather than all, embodiments of the present disclosure. Other embodiments obtained by those of ordinary skill in the art on the basis of the embodiments of the present disclosure without creative efforts all fall within the protection scope of the present disclosure.

Terms used in the embodiments of the present disclosure are for the purpose of describing specific embodiments only and are not intended to limit the present disclosure. The singular forms “a,” “the,” and “said” used in the embodiments and appended claims of the present disclosure are also intended to represent plural forms thereof. Unless otherwise clearly noted in the context, “a plurality of” generally includes at least two, but including at least one should not be excluded.

It should be appreciated that the term “and/or” used herein is merely an association relationship describing associated objects, indicating that there may be three relations. For example, A and/or B may indicate the following three cases: A exists individually, A and B exist simultaneously, and B exists individually. In addition, the character “I” herein generally indicates that the associated objects before and after the character form an “or” relation.

Depending on the context, the term “if” as used herein may be interpreted as “when,” or “in the case that,” or “in response to a determination,” or “in response to a testing”. Similarly, depending on the context, the phrase “if determined” or “if testing (a stated condition or event)” may be interpreted as “when determined” or “in response to a determination,” or “when testing (a stated condition or event)” or “in response to testing (a stated condition or event).”

It should also be noted that the term “comprise,” “include,” or any other variant thereof is intended to encompass a non-exclusive inclusion, so that a product or system that involves a series of elements comprises not only those elements, but also other elements not explicitly listed, or elements that are inherent to such a product or system. Without more restrictions, an element defined by the phrase “comprising a . . . ” does not exclude the presence of another same element in the product or system that comprises the element.

In addition, the sequence of steps in the following method embodiments is only an example and is not to impose a strict limitation.

### Term Definitions

Genetic sequencing: a novel gene testing technology, which can be used to analyze and determine a complete genetic sequence from blood or saliva to predict a possibility of contracting a plurality of diseases, individual behavior characteristics, and behavior rationality. The genetic sequencing technology can be used to lock individual diseased genes, so that prevention and treatment are carried out in advance based on the individual diseased genes.

Mutation analysis: gene mutation is sudden heritable mutation occurring to DNA molecules of a genome. At the molecular level, gene mutation is a change in composition or arrangement of a base pair in a gene structure. Although genes are stable enough to precisely replicate the genes themselves during cell division, such stability is relative. Under some conditions, a gene may also suddenly change from its original existence form to another new existence form. In short, a new gene suddenly appears at a site to replace an original gene.

SNP: single nucleotide polymorphism, which is mainly a DNA sequence polymorphism caused by mutation of a single nucleotide at the genome level. The SNP is the most common human heritable mutation, accounting for more than 90% of all known polymorphisms. SNPs widely exist in human genomes, with an average of one in every 300 base pairs, and it is estimated that a total quantity of the SNPs can reach 3 million or more. SNP is a dimorphic marker, and is caused by conversion or transversion of a single base, or may be caused by insertion or deletion of a base. The SNP may be within a genetic sequence or on a non-coding sequence other than a gene.

Indel: insertion-deletion, translated as an insertion-deletion marker, is a difference between two parents in a complete genome. Relative to the other parent, a certain quantity of nucleotides is inserted into or deleted from a genome of one of the parents. According to insertion-deletion sites in the genome, some polymerase chain reaction PCR primers are designed to amplify the insertion-deletion sites, and therefore, are called Indel markers.

Reads: a DNA fragment with a specific length. The length depends on a reading length of a sequencer.

Deep learning: referring to learning inherent laws and representation levels of sample data. Information obtained during such learning processes is of great help to interpretation of data such as a text, an image, and a sound. The ultimate goal of deep learning is to enable machines to have analytical learning capabilities like humans, and to recognize data such as a text, an image, and a sound.

A sequencing depth: an average number of times that a single base on a sequenced genome has been sequenced. For example, a sequencing depth of a sample is 30×, which indicates that each single base on a genome of the sample is sequenced (or read) 30 times on average. Of course, sequencing depths also have maximum and minimum values that are obtained through information analysis. In fact, to improve precision, a sequencing depth is generally 15×.

Convolutional Neural Networks (CNN for short): a type of Feedforward Neural Networks that include convolution computation and have a deep structure, and are one of representative algorithms of deep learning.

Generative Adversarial Networks (GAN for short): a type of deep learning model, and one of promising methods for unsupervised learning on complicated distributions in recent years. The model produces a fairly good output through mutual game learning of (at least) two modules in a framework: a generative model and a discriminative model.

To understand specific implementation processes of the technical solutions in the embodiments, related technologies are described below.

For humans, a reads fragment covers 23 pairs of chromosomes, totaling more than 3 billion base pairs. Information in each reads fragment may include: a base sequence, a quality sequence, positive and negative chains, and the like. The foregoing base sequence is in a one-to-one correspondence with the quality sequence. At this point, how to effectively use the massive sequencing information and test a mutation site and a related attribute of mutation is a challenging task.

Generally, it costs tens of thousands of yuan to do a complete genome sequencing. With continuous development of sequencing technologies in recent years, costs of genetic sequencing have been reduced to a certain extent. However, genetic sequencing still costs a lot. Therefore, how to reduce genetic testing costs is an urgent problem to be resolved.

Sequencing price is strictly positively correlated with a depth of sequencing data. Therefore, if high-accuracy mutation identification may still be implemented for a low-depth sequencing result from a perspective of a sequencing depth, costs will be greatly reduced. For example, if the precision of a mutation analysis algorithm for 20-time-depth data can be made to be equivalent to the precision for 40-time-depth data, sequencing costs may be halved.

At present, a genetic testing method in the prior art includes obtaining low-depth gene data, extracting a signature by using a linear model Clair, obtaining a low-depth signature, performing testing based on the low-depth signature, and obtaining a genetic testing result. During the signature extraction, a small-size image in a pileup format is used. In this method, sparse information of all reads fragments may be statistically integrated. For example, all the information may be stored in a three-dimensional array, and the three dimensions respectively represent: location information centered on a candidate location (for example, a data length is 33), positive and negative chains corresponding to four different bases (A, G, C, T, A-, G-, C-, T-), and four pieces of different statistical information (statistics that are the same as those of a reference base, statistics of base insertion, statistics of base deletion, and different statistics of a single base).

The signature extraction manner using Clair requires less calculation, achieves a faster speed and higher operation efficiency, and results in lower costs of genetic testing. However, the foregoing genetic testing result is obtained through analysis of a low-depth signature. That is, the low-depth signature extracted by using the linear model Clair is not complete enough, thereby reducing the accuracy of performing data analysis and processing based on a gene signature and failing to meet a genetic sequencing requirement.

To resolve the foregoing technical problems, the embodiments put forward a genetic testing method, a signature extraction method, an apparatus, and a device. The foregoing genetic testing method may be executed by a genetic testing terminal, and a genetic sequence collection terminal may be disposed on the genetic testing terminal. Alternatively, the genetic testing terminal may be in a communication connection with the genetic sequence collection terminal.

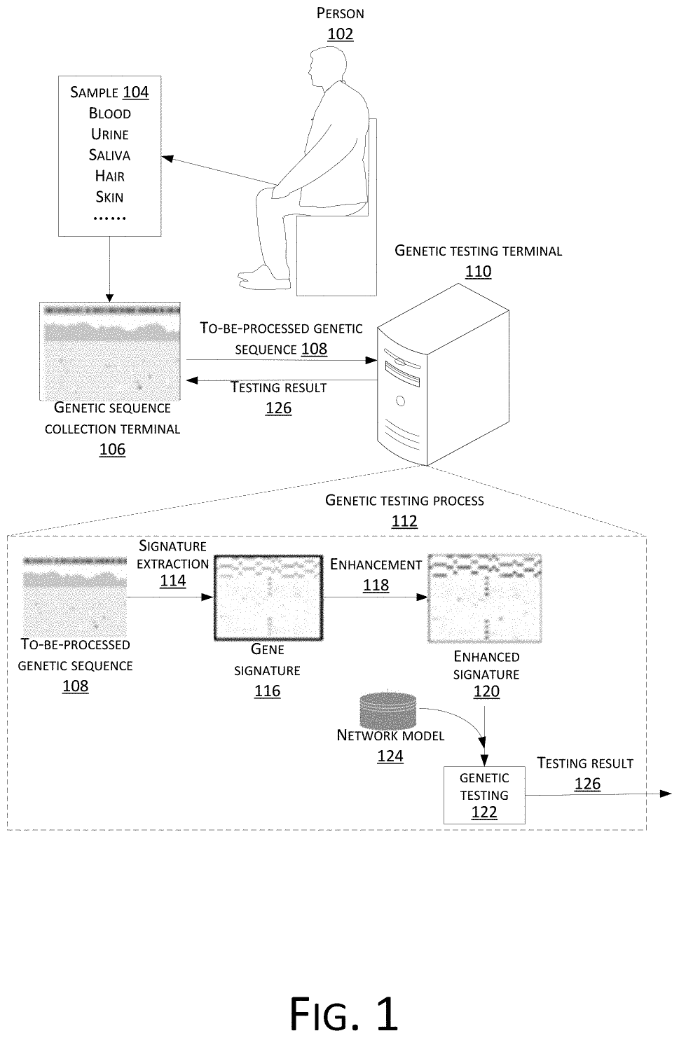

Refer to FIG. 1, a person 102's sample 104 is collected by a genetic sequence collection terminal 106. The sample 104 may be blood, urine, salvia, hair, skin, or any other piece of the human body of the person 102 that include genetic sequence. The genetic sequence collection terminal 106 obtains a to-be-processed genetic sequence 108 from the sample 104, and sends the to-be-processed genetic sequence 108 to a genetic testing terminal 110. For example, the average number of gene fragments corresponding to each position in the to-be-processed genetic sequence 108 is less than or equal to a preset threshold. The genetic testing terminal 110 performs a genetic testing process 112 which may include the following acts. The genetic testing terminal 110 performs signature extraction 114 on the to-be-processed genetic sequence 108 to obtain a gene signature 116. The genetic testing terminal 110 conducts enhancement 118 of the gene signature 116 to obtain an enhanced signature 120 corresponding to the gene signature 116. The genetic testing terminal 110 conducts a genetic testing 122 by inputting the enhanced signature 120 into a network model 124 such as a three-dimensional network model to obtain a test result 126. The three-dimensional network model is trained to test a genetic sequence based on a gene signature. The genetic testing terminal 110 sends the testing result 126 to the genetic sequence collection terminal 106.

The genetic sequence collection terminal 106 may be any computing device with a genetic sequence transmission capability and a genetic sequence collection capability. During specific implementation, the genetic sequence collection terminal 106 may be a blood collector, a saliva collector, a skin collector, and the like. In addition, a basic structure of the genetic sequence collection terminal may include at least one processor. The number of processors depends on the configuration and the type of the genetic sequence collection terminal. The genetic sequence collection terminal 106 may also include a memory. The memory may be volatile, such as an RAM, or non-volatile, such as a Read-Only Memory (ROM for short) or a flash memory, or may include both types. The memory usually stores an Operating System (OS for short) and one or more application programs, and may also store program data. In addition to the processing unit and the memory, the genetic sequence collection terminal 106 further includes some basic configurations, such as a network interface card chip, an IO bus, a display component, and some peripheral devices. For example, some peripheral devices may include, for example, a keyboard, a mouse, a stylus, and a printer. Other peripheral devices are well known in the art and are not repeated herein.

The genetic testing terminal 110 is a device that may provide a genetic testing service in a virtual network environment, and is usually an apparatus that uses a network to carry out information planning and genetic testing. During physical implementation, the genetic testing terminal 110 may be any device that can provide a computing service, respond to a service request, and perform processing. For example, the genetic testing terminal 110 may be a cluster server, a regular server, a cloud server, a cloud host, a virtual center, or the like. The genetic testing terminal 110 may include a processor, a hard disk, a memory, a system bus, and the like.

In the foregoing embodiment, the genetic sequence collection terminal 110 may establish a network connection to the genetic testing terminal 106, and the network connection may be wireless or wired. If the genetic sequence collection terminal is in a communication connection with the genetic testing terminal, a network format of the mobile network may be any one of 2G (GSM), 2.5G (GPRS), 3G (WCDMA, TD-SCDMA, CDMA2000, and UTMS), 4G (LTE), 4G+(LTE+), WiMax, 5G, and the like.

In this embodiment of this application, the genetic sequence collection terminal 106 may perform collection on a specified object (a person, an animal, or the like), so that a to-be-processed genetic sequence can be obtained. An average number of gene fragments corresponding to each position in the genetic sequence is less than or equal to a preset threshold, that is, the to-be-processed genetic sequence is low-depth genetic sequence data. After the to-be-processed genetic sequence is obtained, the genetic sequence collection terminal 106 may upload the to-be-processed genetic sequence to the genetic testing terminal 110, so that the genetic testing terminal 110 may analyze and process the uploaded to-be-processed genetic sequence.

The genetic testing terminal 110 is configured to receive the to-be-processed genetic sequence uploaded by the genetic sequence collection terminal, and then the genetic testing terminal 110 may perform signature extraction on the genetic sequence, so that a gene signature of the genetic sequence may be obtained. The genetic sequence is low-depth data. Therefore, the obtained gene signature is a low-depth signature. To improve genetic testing precision, after the gene signature is obtained, the gene signature may be enhanced, and an enhanced signature corresponding to the gene signature is obtained. The enhanced signature is a high-depth signature or similar to a high-depth signature. After the enhanced signature is obtained, the genetic sequence may be tested based on the enhanced signature, so that the testing result 126 may be precisely and effectively obtained.

According to the technical solution provided in this embodiment, signature extraction is performed on the low-depth genetic sequence, the low-depth gene signature is obtained, the gene signature is enhanced, the enhanced signature is obtained, and then testing is performed based on the enhanced signature, thereby not only ensuring genetic testing precision, but also further effectively reducing data processing costs and a quantity of processed data, and further improving practicality of the method.

Some implementation manners of the present disclosure are described below in detail with reference to the accompanying drawings. As long as no conflicts between the embodiments are caused, the embodiments and the signatures in the embodiments below may be combined with one another.

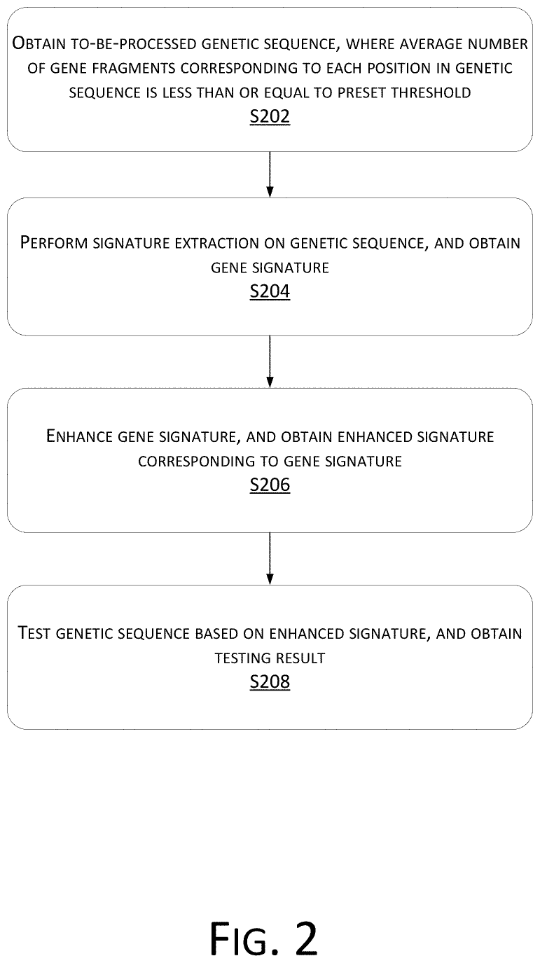

FIG. 2 is a schematic flowchart of a genetic testing method according to an embodiment of the present disclosure. Referring to FIG. 2, this embodiment provides a genetic testing method, and the method may be executed by a genetic testing apparatus. It may be understood that the genetic testing apparatus may be implemented as software or a combination of software and hardware. For example, the genetic testing method may include the following steps.

Step S202: Obtain a to-be-processed genetic sequence, where an average number of gene fragments corresponding to each position in the genetic sequence is less than or equal to a preset threshold.

Step S204: Perform signature extraction on the genetic sequence, and obtain a gene signature.

Step S206: Enhance the gene signature, and obtain an enhanced signature corresponding to the gene signature.

Step S208: Test the genetic sequence based on the enhanced signature, and obtain a testing result.

The foregoing steps are described below in detail.

Step S202: Obtain the to-be-processed genetic sequence, where the average number of gene fragments corresponding to each position in the genetic sequence is less than or equal to the preset threshold.

The to-be-processed genetic sequence is sequence data on which genetic testing needs to be performed. The foregoing genetic testing may include gene characteristic testing, and the gene characteristic testing may include gene stability testing, gene variability testing (that is, gene mutation testing), and the like. For example, in this embodiment, genetic testing may be performed depending on a specific application scenario or application requirement. In addition, each position in the sequence data may correspond to a plurality of gene fragments. The foregoing gene fragment may include a base quality. It may be understood that the gene fragment may include not only the foregoing the base quality, but also other information. For example, the gene fragment may include base information (A, C, G, T), mapping quality, positive and negative chains (A, C, G, T, A-, C-, G-, T-, among which the latter four are negative chains and the former four are positive chains), and other information.

It should be noted that the average number of gene fragments corresponding to each position in the to-be-processed genetic sequence is less than or equal to the preset threshold, that is, the to-be-processed genetic sequence is defined as a low-depth genetic sequence. It may be understood that the preset threshold is an upper limit value that is pre-configured for defining data as low-depth gene data. A specific value range may be adjusted based on different application scenarios or application requirements. For example, the preset threshold may be 10×, 15×, or 20×. For example, when the preset threshold is 15×, when the average number of gene fragments corresponding to each position in the genetic sequence is less than or equal to 15×, it indicates that the genetic sequence is low-depth gene data; when the average number of gene fragments corresponding to each position in the genetic sequence is greater than 15×, it indicates that the genetic sequence is high-depth gene data. To reduce costs required for genetic sequencing, a genetic sequence in which an average number of gene fragments corresponding to each position is less than or equal to the preset threshold is obtained, so that genetic testing may be performed based on a low-depth genetic sequence.

In addition, a specific manner of obtaining the genetic sequence is not limited in this embodiment. For example, the to-be-processed genetic sequence may be stored in a specified region, and the genetic sequence can be obtained by accessing the specified region. In other instances, a gene collection module is disposed on the genetic testing apparatus, and the genetic sequence can be obtained by using the gene collection module. In different application scenarios, the gene collection module may correspond to different structural features. For example, when a to-be-processed genetic sequence is obtained by using blood, the gene collection module may be a blood collector. For example, the blood collector collects blood from a body of a specified object (a person, an animal, or the like) and extracts a to-be-processed genetic sequence based on the blood. Similarly, when a to-be-processed genetic sequence is obtained by using saliva, the gene collection module may be a saliva collector. For example, the saliva collector collects saliva from a body of a specified object (a person, an animal, or the like) and extracts a to-be-processed genetic sequence based on the saliva. Similarly, when a to-be-processed genetic sequence is obtained by using skin, the gene collection module may be a skin collector. For example, the skin collector collects skin from a body of a specified object (a person, an animal, or the like) and extracts a to-be-processed genetic sequence based on the skin.

Apparently, a person skilled in the art may also obtain the to-be-processed genetic sequence in another manner, as long as the accuracy and reliability of obtaining the to-be-processed genetic sequence can be ensured. Details are not described herein.

Step S204: Perform signature extraction on the genetic sequence, and obtain the gene signature.

After the genetic sequence is obtained, signature extraction may be performed on the genetic sequence, and the gene signature is obtained. It should be noted that, because the genetic sequence is a low-depth genetic sequence, the gene signature obtained after signature extraction is performed on the genetic sequence is a low-depth gene signature, and the low-depth gene signature includes a relatively small quantity of information.

Step S206: Enhance the gene signature, and obtain the enhanced signature corresponding to the gene signature.

The gene signature obtained by performing signature extraction on the genetic sequence is the low-depth gene signature, and the low-depth gene signature includes a relatively small quantity of information. Therefore, to improve the genetic testing precision, the gene signature may be enhanced, so that the enhanced signature corresponding to the gene signature may be obtained. The enhanced signature obtained includes a relatively large quantity of information, that is, the enhanced signature is a high-depth signature or similar to a high-depth signature. In this way, the quality and efficiency of genetic testing can be effectively improved when testing is performed based on the enhanced signature.

In some instances, the step of enhancing the gene signature and obtaining the enhanced signature corresponding to the gene signature in this embodiment may include obtaining a convolutional neural network model for enhancing the gene signature; and enhancing the gene signature based on the convolutional neural network model, and obtaining the enhanced signature corresponding to the gene signature.

A convolutional neural network for enhancing the gene signatures is pre-configured, the convolutional neural network may be a fully convolutional neural network, and the convolutional neural network may be a two-dimensional network model or a three-dimensional network model. For example, after the gene signature is obtained, the gene signature may be input into the convolutional neural network model, so that the gene signature may be enhanced based on the convolutional neural network model, and the enhanced signature corresponding to the gene signature may be obtained. The quantity of information included in the enhanced signature obtained is greater than the quantity of information included in the gene signature. In addition, a data magnitude of the enhanced signature obtained may be the same as a data magnitude of the gene signature, thereby facilitating testing performed based on the enhanced signature, and further improving quality and efficiency of testing.

Step S208: Test the genetic sequence based on the enhanced signature, and obtain the testing result.

After the enhanced signature is obtained, the genetic sequence may be tested based on the enhanced signature, and the testing result is obtained. In this embodiment, a specific implementation manner of testing the genetic sequence based on the enhanced signature is not limited, and a person skilled in the art may perform setting depending on a specific application scenario or application requirement. In some instances, the step of testing the genetic sequence based on the enhanced signature and obtaining the testing result may include inputting the enhanced signature into the three-dimensional network model, and obtaining the testing result. The three-dimensional network model is trained to test a genetic sequence based on a gene signature.

For example, a three-dimensional network model for testing a genetic sequence is trained in advance. After the enhanced signature is obtained, the enhanced signature may be input into the three-dimensional network model. After the three-dimensional network model obtains the enhanced signature, the enhanced signature may be tested, so that the testing result can be obtained.

In some other instances, when genetic testing can be performed to implement mutation testing, the step of testing the genetic sequence based on the enhanced signature and obtaining the testing result in this embodiment may include: obtaining, based on the enhanced signature, mutation reference information corresponding to the enhanced signature, where the mutation reference information includes at least one of the following: prediction information of 21 genotypes, zygote prediction information, first allele mutation length information, and second allele mutation length information; and obtaining a mutation testing result based on the mutation reference information.

For example, after the enhanced signature is obtained, the enhanced signature is analyzed and processed, so that the mutation reference information corresponding to the enhanced signature may be obtained. The mutation reference information may include at least one of the following: the prediction information of the 21 genotypes, the zygote prediction information, the first allele mutation length information, and the second allele mutation length information. The 21 genotypes targeted by the foregoing prediction information of the 21 genotypes include: ‘AA’, ‘AC’, ‘AG’, ‘AT’, ‘CC’, ‘CG’, ‘CT’, ‘GG’, ‘GT’, ‘TT’, ‘AI’, ‘CI’, ‘GI’, ‘TI’, ‘AD’, ‘CD’, ‘GD’, ‘TD’, ‘II’, and ‘DD’. A, C, G, and T are four bases, and I and D are respectively insertion and deletion. The foregoing zygote prediction information includes three cases: a zygote is a homozygote and is consistent with a reference base, the zygote is a homozygote and is inconsistent with the reference base, and the zygote is a heterozygote. For the first allele mutation length information, an SNP mutates to 0, and Indel mutation is insertion of a deleted length correspondingly. For the second allele mutation length information, an SNP mutates to 0, and Indel mutation is insertion of a deleted length correspondingly.

After the mutation reference information corresponding to the enhanced signature is obtained, the mutation reference information may be analyzed and processed to obtain the mutation testing result. It may be understood that the mutation testing result is obtained based on at least one of the prediction information of the 21 genotypes, the zygote prediction information, the first allele mutation length information, and the second allele mutation length information, thereby ensuring the accuracy and reliability of determining the mutation testing result.

In still some other instances, after the mutation testing result is obtained, the method in this embodiment may further include performing disease prediction based on the mutation testing result.

When a mutation exists in the genetic sequence, it indicates that a specified object is relatively prone to a related disease. In other words, a probability of producing a related disease is relatively high. At this point, disease prediction may be performed based on the mutation testing result. For example, the probability that the specified object produces a related disease may be determined based on the mutation in the genetic sequence. It may be understood that the probability is correlated with a degree of the mutation in the genetic sequence. A higher degree of the mutation leads to a higher probability; and a lower degree of the mutation leads to a lower probability. Conversely, when no mutation exists in the genetic sequence, it indicates that the specified object is not prone to a related disease.

In the genetic testing method provided by this embodiment, the to-be-processed genetic sequence is obtained, signature extraction is performed on the genetic sequence, and the gene signature is obtained. The genetic sequence needing to be processed is low-depth gene data. Therefore, the gene signature obtained by performing signature extraction on the low-depth genetic sequence is also a low-depth gene signature, and then the gene signature is enhanced, so that the enhanced signature corresponding to the gene signature may be obtained. The enhanced signature is a high-depth signature or similar to a high-depth signature. Then, the genetic sequence is tested based on the enhanced signature, and the testing result is obtained. In this way, not only is genetic testing precision ensured, but also data processing costs and a quantity of processed data are further effectively reduced, thereby effectively achieving relatively precise testing based on the low-depth gene data, further improving practicality of the method, and facilitating promotion and application in the market.

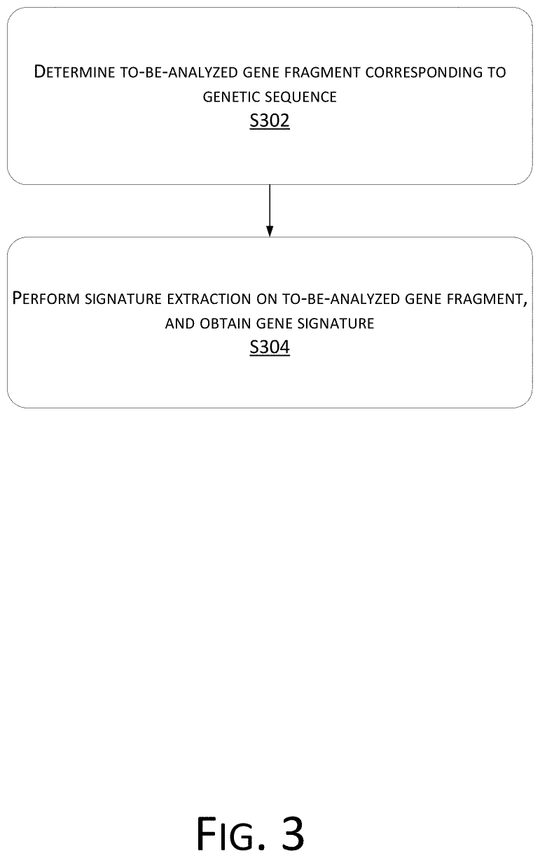

FIG. 3 is a schematic flowchart of performing signature extraction on a genetic sequence and obtaining a gene signature according to an embodiment of the present disclosure. Based on the foregoing embodiment, referring to FIG. 3, this embodiment provides an implementation manner of performing signature extraction on a genetic sequence. For example, performing signature extraction on the genetic sequence and obtaining the gene signature in this embodiment may include the following steps.

Step S302: Determine a to-be-analyzed gene fragment corresponding to the genetic sequence.

After the genetic sequence is obtained, the genetic sequence may be analyzed and processed to determine the to-be-analyzed gene fragment corresponding to the genetic sequence. In some instances, the step of determining a to-be-analyzed gene fragment corresponding to the genetic sequence may include: obtaining reference data and a plurality of initial gene fragments included in the genetic sequence; performing matching between the reference data and the genetic sequence, to determine the to-be-analyzed gene fragment among the plurality of initial gene fragments, where there is a base in the to-be-analyzed gene fragment and that does not match the reference data, and a proportion of the unmatched base in the to-be-analyzed gene fragment is greater than a preset base threshold.

For example, the reference data is standard gene data used to test whether the initial gene fragment is the to-be-analyzed gene fragment, and the plurality of initial gene fragments are gene data that needs to be tested whether they are the to-be-analyzed gene fragments. After the plurality of initial gene fragments and the reference data are obtained, analysis and matching may be performed on the reference data and the plurality of initial gene fragments to determine the to-be-analyzed gene fragment among the plurality of initial gene fragments. For example, the to-be-analyzed gene fragment is at least a part of the plurality of initial gene fragments. It should be noted that there is a base that is in the determined to-be-analyzed gene fragment and that does not match the reference data, and a proportion of the unmatched base in the initial gene fragment is greater than the preset threshold.

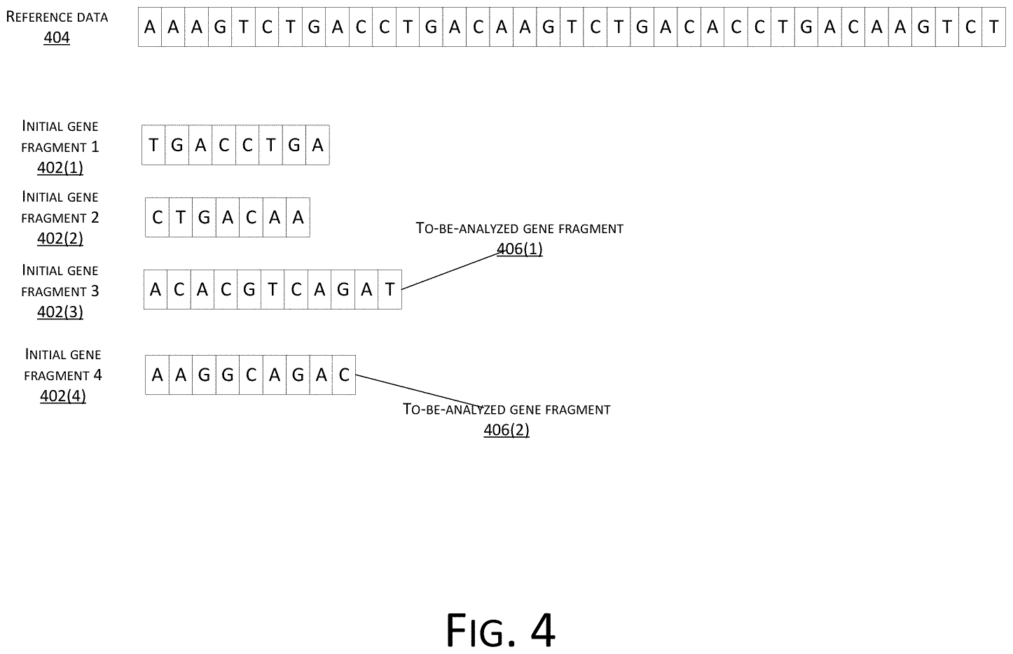

For example, referring to FIG. 4, an example in which the number of the plurality of initial gene fragments 402 included in the genetic sequence is 4, and the reference data 404 AAAGTCTGACCTGACAAGTCTGACACCTGACAAGTCT is used for description. The initial gene fragments may include: an initial gene fragment 1 402(1), an initial gene fragment 2 402(2), an initial gene fragment 3 402(3), and an initial gene fragment 4 402(4). The initial gene fragment 1 402(1) may be TGACCTGA, the initial gene fragment 2 402(2) may be CTGACAA, the initial gene fragment 3 402(3) may be ACACGTCAGAT, and the initial gene fragment 4 402(4) may be AAGGCAGAC.

To improve the genetic testing effectiveness, the foregoing initial gene fragments 402 may be preliminarily screened to preliminarily screen out a gene fragment with an abnormality among the initial gene fragments. For example, the reference data 404 and the initial gene fragments 402 may be analyzed and compared. That is, after the reference data 404 and the initial gene fragment 1 402(1) are obtained, analysis and matching may be performed on the reference data 404 and the initial gene fragment 1 402(1), and the initial gene fragment 1 402(1) matches 12th to 19th bases in the reference data. In other words, bases in the initial gene fragment 1 402(1) completely match the bases in the reference data. At this point, it indicates that no gene abnormality exists in the initial gene fragment 1 402(1), thereby further indicating that the initial gene fragment 1 402(1) does not meet a condition of a to-be-analyzed gene fragment. Therefore, the initial gene fragment 1 402(1) is not determined as the to-be-analyzed gene fragment 406.

After the reference data and the initial gene fragment 2 402(2) are obtained, analysis and matching may be performed on the reference data and the initial gene fragment 2 402(2), and the initial gene fragment 2 402(2) matches 11th to 17th bases in the reference data. In other words, bases in the initial gene fragment 2 402(2) completely match the bases in the reference data. At this point, it indicates that no gene abnormality exists in the initial gene fragment 2 402(2), thereby further indicating that the initial gene fragment 2 402(2) does not meet the condition of the to-be-analyzed gene fragment. Therefore, the initial gene fragment 2 402(2) is not determined as the to-be-analyzed gene fragment.

After the reference data and the initial gene fragment 3 402(3) are obtained, analysis and matching may be performed on the reference data 404 and the initial gene fragment 3 402(3), and the initial gene fragment 3 402(3) partially matches 14th to 24th bases in the reference data 404. In other words, bases in the initial gene fragment 3 402(3) do not completely match the bases in the reference data 404. At this point, it indicates that a gene abnormality exists in the initial gene fragment 3 402(3), the number of unmatched bases is 3, and the total number of bases included in the initial gene fragment 3 402(3) is 11. At this point, a proportion of the unmatched bases in the initial gene fragment 3 402(3) is 3/11, approximately 0.273. Assuming that the preset threshold is 0.1, the proportion of the unmatched bases in the initial gene fragment 3 402(3) is greater than the preset threshold, indicating that the initial gene fragment 3 402(3) meets the condition of the to-be-analyzed gene fragment 406(1), so that the initial gene fragment 3 402(3) may be determined as the to-be-analyzed gene fragment 406(1).

After the reference data and the initial gene fragment 4 402(4) are obtained, analysis and matching may be performed on the reference data and the initial gene fragment 4 402(4), and the initial gene fragment 4 402(4) partially matches 2nd to 10th bases in the reference data 404. In other words, bases in the initial gene fragment 4 402(4) do not completely match the bases in the reference data 404. At this point, it indicates that a gene abnormality exists in the initial gene fragment 4 402(4), the number of unmatched bases is 2, and the total number of bases included in the initial gene fragment is 9. At this point, a proportion of the unmatched bases in the initial gene fragment 4 402(4) is 2/9, approximately 0.222. Assuming that the preset threshold is 0.1, the proportion of the unmatched bases in the initial gene fragment 4 402(4) is greater than the preset threshold, indicating that the initial gene fragment 4 402(4) meets the condition of the to-be-analyzed gene fragment 406(2), so that the initial gene fragment 4 402(4) may be determined as the to-be-analyzed gene fragment 406(2).

In this embodiment, the reference data 404 and the plurality of initial gene fragments 402 are obtained, and then matching is performed on the reference data 404 and the plurality of initial gene fragments 402 to determine the to-be-analyzed gene fragment 406 among the plurality of initial gene fragments, thereby effectively obtaining the to-be-analyzed gene fragment 406 by preliminary screening the initial gene fragments 402. In this way, not only are the accuracy and reliability of determining the to-be-analyzed gene fragment 406 ensured, but also the quality and efficiency of analyzing and processing the gene fragment are improved.

Step S304: Perform signature extraction on the to-be-analyzed gene fragment, and obtain the gene signature.

After the to-be-analyzed gene fragment is obtained, signature extraction may be performed on the to-be-analyzed gene fragment, so that the gene signature may be obtained. In some instances, the step of performing signature extraction on the to-be-analyzed gene fragment and obtaining the gene signature may include: obtaining a base quality included in the to-be-analyzed gene fragment; determining, based on the base quality, a confidence level corresponding to the to-be-analyzed gene fragment; and performing signature extraction on the to-be-analyzed gene fragment based on the confidence level corresponding to the to-be-analyzed gene fragment, and obtaining the gene signature.

For example, the to-be-analyzed gene fragment includes the base quality, and after the to-be-analyzed gene fragment is obtained, information extraction may be performed on the to-be-analyzed gene fragment, so that the base quality included in the to-be-analyzed gene fragment may be obtained. A mapping relationship exists between the base quality and the confidence level that is corresponding to the gene fragment. Therefore, after the base quality included in the to-be-analyzed gene fragment is obtained, the confidence level corresponding to the to-be-analyzed gene fragment may be determined based on the base quality included in the to-be-analyzed gene fragment. In some instances, the step of determining, based on the base quality, a confidence level corresponding to the to-be-analyzed gene fragment may include obtaining a ratio between the base quality and 10; and determining, based on the ratio, the confidence level corresponding to the to-be-analyzed gene fragment. The confidence level is positively correlated with the base quality, and the confidence level is less than 1.

When the quality qual of the base is obtained, the ratio

$$\frac{qual}{10}$$

between the quality qual of the base and 10 may be obtained, and then the confidence levelpcorresponding to the to-be-analyzed gene fragment is determined based on the ratio

$$\frac{qual}{10} .$$

In some instances, the confidence level is

$$p = 1 - 1 {0}^{- \frac{qual}{10}} .$$

At this point, the confidence level pis a value between 0 and 1, and the confidence level p is positively correlated with the base quality. In other words, a larger the base quality leads to a larger the base quality included in the to-be-analyzed gene fragment. At this point, it indicates that the accuracy of the to-be-analyzed gene fragment is higher, so that it may be determined that the confidence level p of the gene fragment also increases. Similarly, a smaller the base quality leads to a smaller confidence level p.

Certainly, a person skilled in the art may also obtain, in another manner, the confidence level p corresponding to the to-be-analyzed gene fragment. For example, the confidence level is

$$p = 1 {0}^{\frac{- qual}{10}} .$$

At this point, the confidence level is negatively correlated with the base quality. That is, a larger the base quality leads to a smaller confidence level p; a smaller the base quality leads to a larger confidence level p.

Further, after the confidence level corresponding to the to-be-analyzed gene fragment is obtained, signature extraction may be performed on the to-be-analyzed gene fragment based on the confidence level corresponding to the to-be-analyzed gene fragment, so that the gene signature of the to-be-analyzed gene fragment may be obtained. In some instances, the step of performing signature extraction on the to-be-analyzed gene fragment based on the confidence level corresponding to the to-be-analyzed gene fragment, and obtaining the gene signature of the to-be-analyzed gene fragment may include: performing signature extraction on the to-be-analyzed gene fragment based on the confidence level corresponding to the to-be-analyzed gene fragment and through statistical counting, and obtaining the gene signature of the to-be-analyzed gene fragment. The gene signature includes base information, a base position, and statistics corresponding to the base information.

For example, the base information may include at least one of the following: A, G, C, T, A-, G-, C-, and T-. The foregoing base information (A, G, C, T) is a positive chain, the base information (A-, G-, C-, T-) is a negative chain, and the statistics corresponding to the base information may include at least one of the following: statistics that are the same as those of a reference base, statistics of base insertion, statistics of base deletion, and different statistics of a single base. After the confidence level corresponding to the to-be-analyzed gene fragment is obtained, signature extraction may be performed on the to-be-analyzed gene fragment based on the confidence level corresponding to the to-be-analyzed gene fragment and through statistical counting, to stably obtain the gene signature of the to-be-analyzed gene fragment in combination with the confidence level corresponding to the to-be-analyzed gene fragment, thereby improving the integrity and efficiency of extracting the gene signature.

In the technical solution provided by this embodiment, the to-be-analyzed gene fragment corresponding to the genetic sequence is determined, then signature extraction is performed on the to-be-analyzed gene fragment, and the gene signature is obtained, thereby effectively achieving the quality and efficiency of extracting the gene signature. For example, in the method, the base quality is effectively integrated into the gene signature on the basis of no increase of data dimensions. In this way, not only is the implementation manner simple and reliable, which also ensures the integrity of extracting the gene signature, but also the operating efficiency of extracting the gene signature is further improved, thereby further improving the practicability of the technical solution.

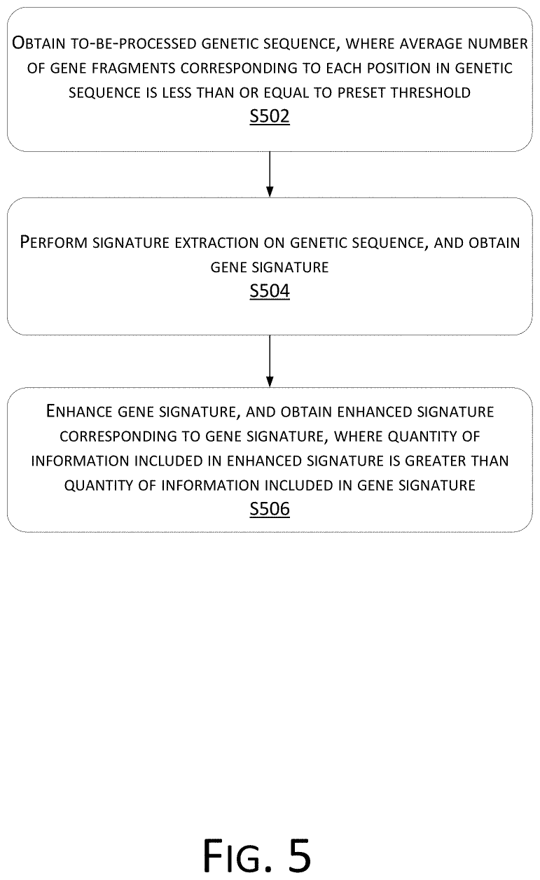

FIG. 5 is a schematic flowchart of a signature extraction method according to an embodiment of the present disclosure. Referring to FIG. 5, this embodiment provides a signature extraction method, and the signature extraction method is executed by a signature extraction apparatus. It may be understood that the signature extraction apparatus may be implemented as software or a combination of software and hardware. For example, the signature extraction method may include the following steps.

Step S502: Obtain a to-be-processed genetic sequence, where an average number of gene fragments corresponding to each position in the genetic sequence is less than or equal to a preset threshold.

Step S504: Perform signature extraction on the genetic sequence, and obtain a gene signature.

Step S506: Enhance the gene signature, and obtain an enhanced signature corresponding to the gene signature, where a quantity of information included in the enhanced signature is greater than a quantity of information included in the gene signature.

For example, a specific implementation process and a specific implementation effect of the foregoing steps in this embodiment are similar to the specific implementation process and the specific implementation effect of the steps S202 to S206 in the foregoing embodiment. Reference may be made to the foregoing statements, and details are not described herein again.

In the signature extraction method provided by this embodiment, the to-be-processed genetic sequence is obtained, signature extraction is performed on the genetic sequence, and the gene signature is obtained. The obtained genetic sequence is low-depth gene data. Therefore, the gene signature obtained by performing signature extraction on the low-depth genetic sequence is also a low-depth gene signature, and then the gene signature is enhanced, so that the enhanced signature corresponding to the gene signature may be obtained. Then, the genetic sequence is tested based on the enhanced signature, and the testing result is obtained. In this way, not only is genetic testing precision ensured, but also data processing costs and a quantity of processed data are further effectively reduced, thereby effectively achieving relatively precise testing based on the low-depth gene data, further improving practicality of the method, and facilitating promotion and application in the market.

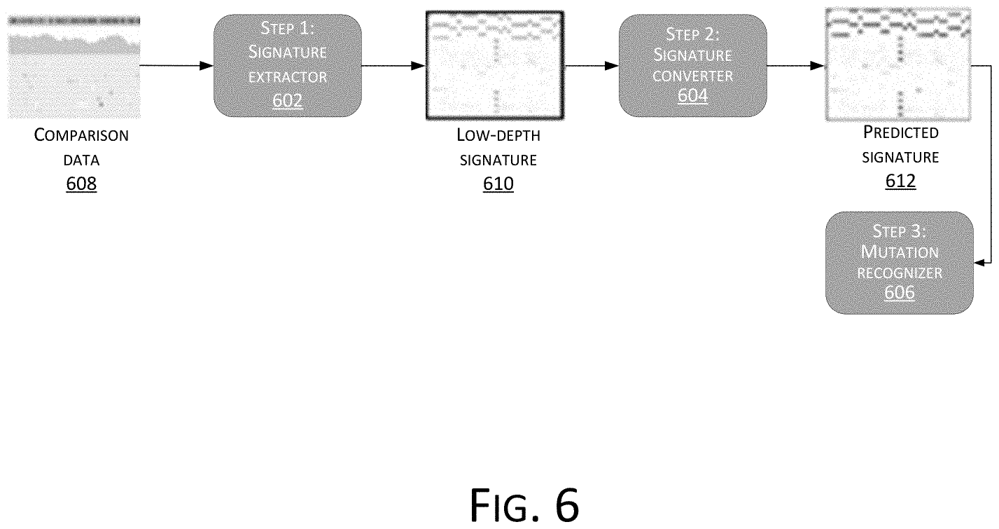

During specific applications, referring to FIG. 6, the application embodiment provides a gene mutation testing method. The gene mutation testing method may be executed by a gene mutation testing apparatus, and the gene mutation testing apparatus may include a signature extractor 602, a signature converter 604, and a mutation recognizer 606. When the gene mutation testing apparatus executes the gene mutation testing method, the following steps may be included.

Step 1: Obtain comparison data 608, where the comparison data is low-depth gene data.

Step 2: Perform signature extraction on the comparison data 608, and obtain a low-depth signature 610.

For example, after the comparison data 608 is obtained, signature extraction may be performed on the comparison data 608 by using the signature extractor 602, and the low-depth signature 610 corresponding to the comparison data 608 is obtained.

Step 3: Perform signature enhancement on the low-depth signature 610, and obtain a predicted signature 612.

After the low-depth signature is obtained, signature enhancement may be performed on the low-depth signature by using the signature converter, and the predicted signature is obtained. The predicted signature is a high-depth signature or similar to a high-depth signature, and the predicted signature may include relatively rich information compared with the low-depth signature. A size of the predicted signature is the same as a size of the low-depth signature.

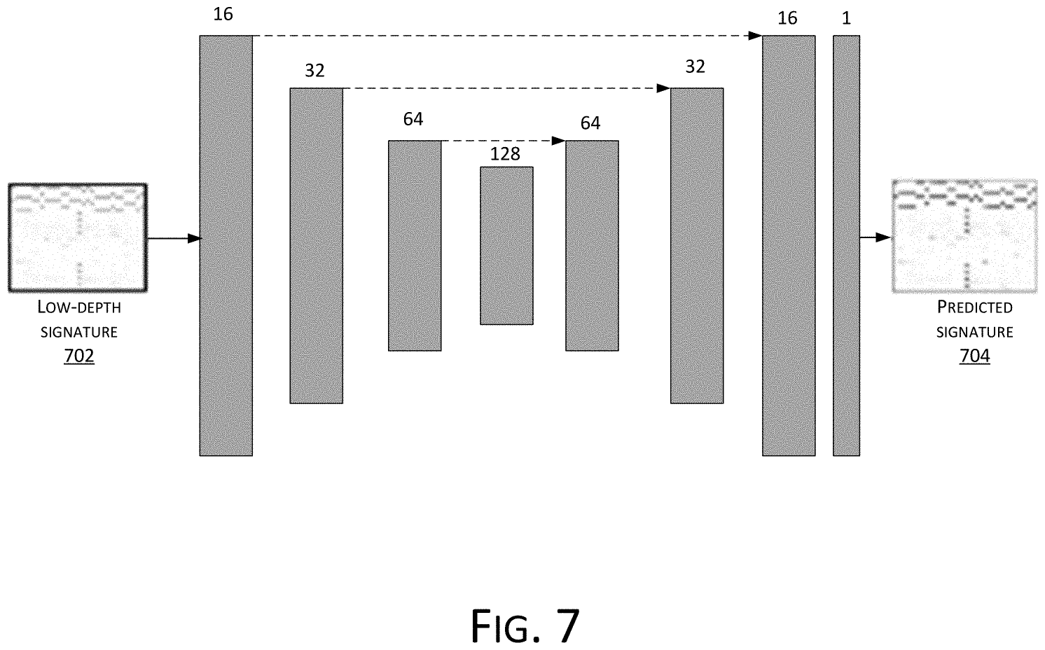

In some instances, referring to FIG. 7, the signature converter may be a two-dimensional fully convolutional neural network model. The foregoing fully convolutional neural network module has learned a correlation between data distribution of low-depth sequencing data and data distribution of high-depth sequencing data. A model structure of the convolutional neural network model may be a U-shaped structure, and may for example include: the number of signature channels (in other words, the number in the figure). A convolution kernel may be 3 or another value. In addition, the arrow in the accompanying drawing indicates that a low-depth signature is integrated into a corresponding high-depth signature. After the low-depth signature is obtained, the low-depth signature 702 may be input into a two-dimensional signature converter, so that the signature converter may perform signature enhancement on the low-depth signature, and a high-depth predicted signature 704 or a predicted signature similar to a high-depth signature may be obtained.

For the signature converter, when a low-depth signature map extracted from the low-depth sequencing data is input, a converted signature map of the same size may be output. The converted signature map is similar to a high-depth signature map, thereby implementing signature conversion from a low depth to a high depth. The low-depth data is processed in the foregoing manner, so that the enhanced signature obtained is closer to high-depth data, thereby finally achieving an effect of reducing sequencing costs.

Step 4: Perform mutation recognition based on the predicted signature, and obtain a mutation recognition result.

After the predicted signature is obtained, the predicted signature may be analyzed and processed by using the mutation recognizer, so that the mutation recognition result may be obtained.

In this embodiment, for a candidate sample position in each piece of comparison data, a sequencing signature at the position is extracted first, and the low-depth signature is mapped to the high-depth signature by using a fully convolutional neural network. Then, mutation testing is performed based on an enhanced high-depth signature, and a mutation testing result is obtained. In this way, not only is gene mutation testing precision ensured, but also data processing costs and a quantity of processed data are further effectively reduced, thereby effectively achieving relatively precise mutation testing based on the low-depth gene data, further improving practicality of the method, and facilitating promotion and application in the market.

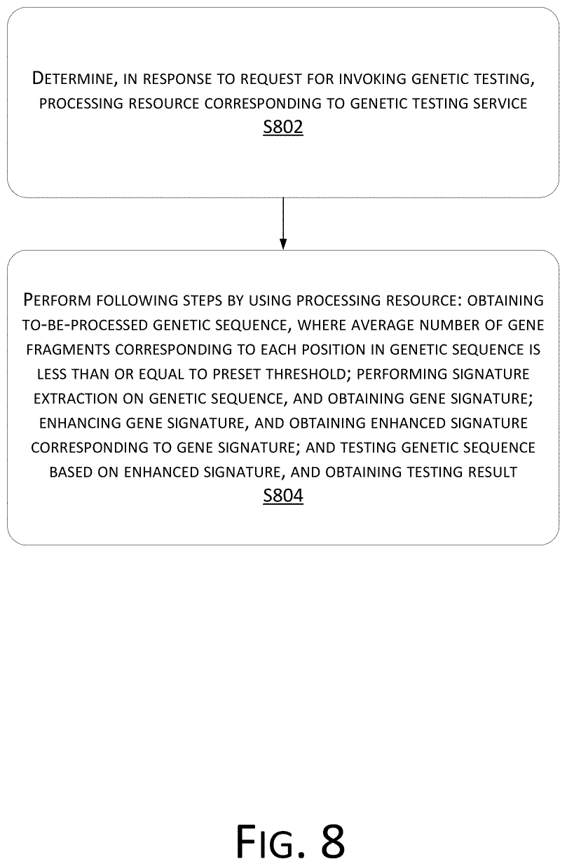

FIG. 8 is a schematic flowchart of a genetic testing method according to an embodiment of the present disclosure. Referring to FIG. 8, this embodiment provides a genetic testing method, and the genetic testing method may be executed by a genetic testing apparatus. It may be understood that the genetic testing apparatus may be implemented as software or a combination of software and hardware. For example, the genetic testing method may include the following steps.

Step S802: Determine, in response to a request for invoking genetic testing, a processing resource corresponding to a genetic testing service.

Step S804: Perform the following steps by using the processing resource: obtaining a to-be-processed genetic sequence, where an average number of gene fragments corresponding to each position in the genetic sequence is less than or equal to a preset threshold; performing signature extraction on the genetic sequence, and obtaining a gene signature; enhancing the gene signature, and obtaining an enhanced signature corresponding to the gene signature; and testing the genetic sequence based on the enhanced signature, and obtaining a testing result.

For example, the genetic testing method provided by the present disclosure may be performed on a cloud, several computing nodes may be deployed on the cloud, and each of the computing nodes has a processing resource, such as a computing resource or a storage resource. On the cloud, a plurality of computing nodes may be organized to provide a certain service. Of course, one computing node may also provide one or more services.

For the solution provided by the present disclosure, the cloud may provide a service for completing the genetic testing method, and the service is referred to as the genetic testing service. When a user needs to use the genetic testing service, the genetic testing service is invoked to trigger a request for invoking the genetic testing service to the cloud. The to-be-processed genetic sequence may be carried in the request. The cloud determines a computing node responding to the request, and performs the following steps by using the processing resource in the computing node: obtaining the to-be-processed genetic sequence, where the average number of gene fragments corresponding to each position in the genetic sequence is less than or equal to the preset threshold; performing signature extraction on the genetic sequence, and obtaining the gene signature; enhancing the gene signature, and obtaining the enhanced signature corresponding to the gene signature; and testing the genetic sequence based on the enhanced signature, and obtaining the testing result.

For example, an implementation process, an implementation principle, and an implementation effect of the foregoing method steps in this embodiment are similar to the implementation process, the implementation principle, and the implementation effect of the method steps in the embodiments shown in FIG. 1 to FIG. 4, FIG. 6, and FIG. 7. For content not described in detail in this embodiment, reference may be made to the related descriptions of the embodiments shown in FIG. 1 to FIG. 4, FIG. 6, and FIG. 7.

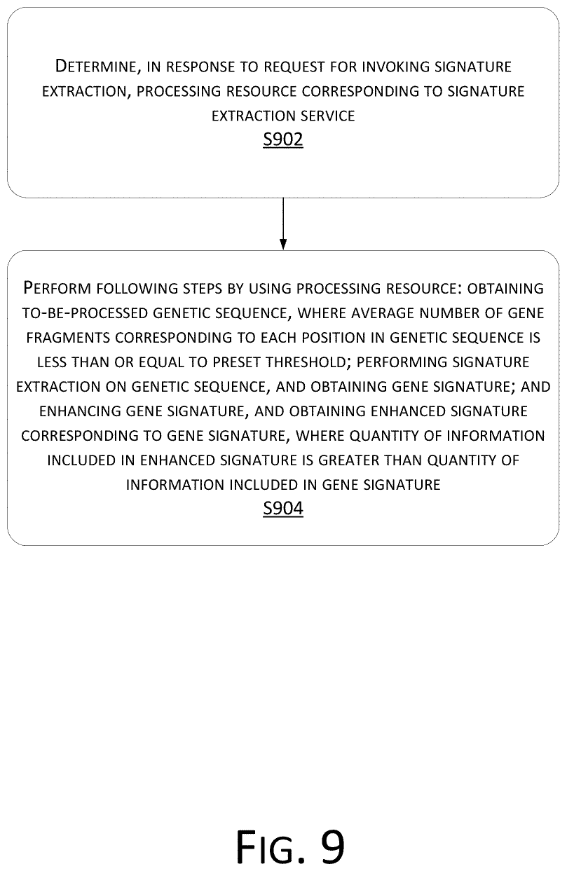

FIG. 9 is a schematic flowchart of a signature extraction method according to an embodiment of the present disclosure. Referring to FIG. 9, this embodiment provides a signature extraction method, and the signature extraction method may be executed by a signature extraction apparatus. It may be understood that the signature extraction apparatus may be implemented as software or a combination of software and hardware. For example, the signature extraction method may include the following steps.

Step S902: Determine, in response to a request for invoking signature extraction, a processing resource corresponding to a signature extraction service.

Step S904: Perform the following steps by using the processing resource: obtaining a to-be-processed genetic sequence, where an average number of gene fragments corresponding to each position in the genetic sequence is less than or equal to a preset threshold; performing signature extraction on the genetic sequence, and obtaining a gene signature; and enhancing the gene signature, and obtaining an enhanced signature corresponding to the gene signature, where a quantity of information included in the enhanced signature is greater than a quantity of information included in the gene signature.

For example, the signature extraction method provided by the present disclosure may be performed on a cloud, several computing nodes may be deployed on the cloud, and each of the computing nodes has a processing resource, such as a computing resource or a storage resource. On the cloud, a plurality of computing nodes may be organized to provide a certain service. Of course, one computing node may also provide one or more services.

For the solution provided by the present disclosure, the cloud may provide a service for completing the signature extraction method, and the service is referred to as the signature extraction service When a user needs to use the signature extraction service, the signature extraction service is invoked to trigger a request for invoking the signature extraction service to the cloud. The to-be-processed genetic sequence may be carried in the request. The cloud determines a computing node that responds to the request, and performs the following steps by using the processing resource in the computing node: obtaining the to-be-processed genetic sequence, where the average number of gene fragments corresponding to each position in the genetic sequence is less than or equal to the preset threshold; performing signature extraction on the genetic sequence, and obtaining the gene signature; and enhancing the gene signature, and obtaining the enhanced signature corresponding to the gene signature, where the quantity of information included in the enhanced signature is greater than the quantity of information included in the gene signature.

For example, an implementation process, an implementation principle, and an implementation effect of the foregoing method steps in this embodiment are similar to the implementation process, the implementation principle, and the implementation effect of the method steps in the embodiments shown in FIG. 5 to FIG. 7. For content not described in detail in this embodiment, reference may be made to the related descriptions of the embodiments shown in FIG. 5 to FIG. 7.

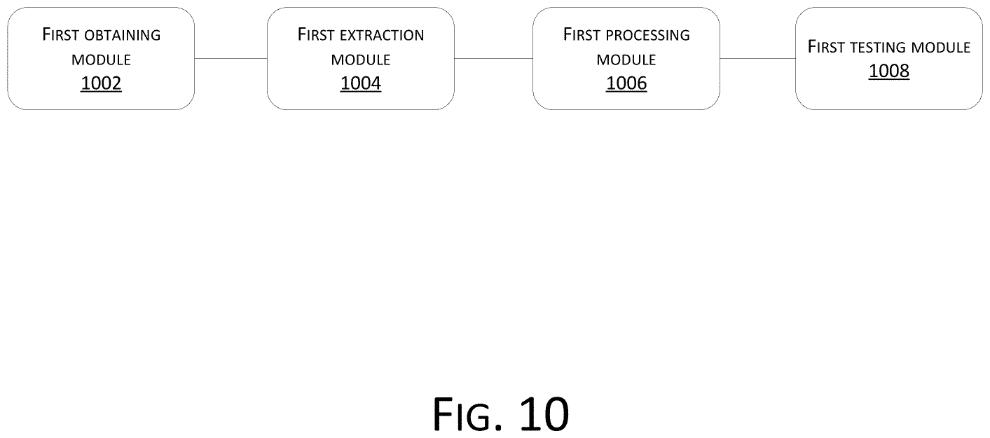

FIG. 10 is a schematic structural diagram of a genetic testing apparatus according to an embodiment of the present disclosure. Referring to FIG. 10, this embodiment provides a genetic testing apparatus, the genetic testing apparatus may perform the foregoing genetic testing method shown in FIG. 2, and the genetic testing apparatus may include: a first obtaining module 1002, a first extraction module 1004, a first processing module 1006, and a first testing module 1008. For example,

the first obtaining module 1002 is configured to obtain a to-be-processed genetic sequence, where an average number of gene fragments corresponding to each position in the genetic sequence is less than or equal to a preset threshold;

the first extraction module 1004 is configured to perform signature extraction on the genetic sequence, and obtain a gene signature;

the first processing module 1006 is configured to enhance the gene signature, and obtain an enhanced signature corresponding to the gene signature; and

the first testing module 1008 is configured to test the genetic sequence based on the enhanced signature, and obtain a testing result.

In some instances, when the first extraction module 1004 performs signature extraction on the genetic sequence and obtains the gene signature, the first extraction module 1004 is configured to: determine a to-be-analyzed gene fragment corresponding to the genetic sequence; and perform signature extraction on the to-be-analyzed gene fragment, and obtain the gene signature.

In some instances, when the first extraction module 1004 determines the to-be-analyzed gene fragment corresponding to the genetic sequence, the first extraction module 1004 is configured to: obtain reference data and a plurality of initial gene fragments included in the genetic sequence; and perform matching between the reference data and the genetic sequence, to determine the to-be-analyzed gene fragment among the plurality of initial gene fragments. There is a base in the to-be-analyzed gene fragment and that does not match the reference data, and a proportion of the unmatched base in the to-be-analyzed gene fragment is greater than a preset base threshold.

In some instances, when the first extraction module 1004 performs signature extraction on the to-be-analyzed gene fragment and obtains the gene signature, the first extraction module 1004 is configured to: obtain a the base quality included in the to-be-analyzed gene fragment; determine, based on the base quality, a confidence level corresponding to the to-be-analyzed gene fragment; and perform signature extraction on the to-be-analyzed gene fragment based on the confidence level corresponding to the to-be-analyzed gene fragment, and obtain the gene signature.

In some instances, when the first processing module 1006 enhances the gene signature and obtains the enhanced signature corresponding to the gene signature, the first processing module 1006 is configured to: obtain a convolutional neural network model for enhancing the gene signature; and enhance the gene signature based on the convolutional neural network model, and obtain the enhanced signature corresponding to the gene signature.

In some instances, a quantity of information included in the enhanced signature obtained is greater than a quantity of information included in the gene signature.

In some instances, a data magnitude of the enhanced signature is the same as a data magnitude of the gene signature.

In some instances, when the first testing module 1008 tests the genetic sequence based on the enhanced signature and obtains the testing result, the first testing module 1008 is configured to: obtain, based on the enhanced signature, mutation reference information corresponding to the enhanced signature, where the mutation reference information includes at least one of the following: prediction information of 21 genotypes, zygote prediction information, first allele mutation length information, and second allele mutation length information; and obtain a mutation testing result based on the mutation reference information.

In some instances, when the first testing module 1008 tests the genetic sequence based on the enhanced signature and obtains the testing result, the first testing module 1008 is configured to input the enhanced signature into a three-dimensional network model, and obtain the testing result. The three-dimensional network model is trained to test the genetic sequence based on the gene signature.

The apparatus shown in FIG. 10 may execute the methods in the embodiments shown in FIG. 1 to FIG. 4, FIG. 6, and FIG. 7. For content not described in detail in this embodiment, reference may be made to the related descriptions of the embodiments shown in FIG. 1 to FIG. 4, FIG. 6, and FIG. 7. For an execution process and a technical effect of the technical solution, refer to the descriptions in the embodiments shown in FIG. 1 to FIG. 4, FIG. 6, and FIG. 7. Details are not described herein again.

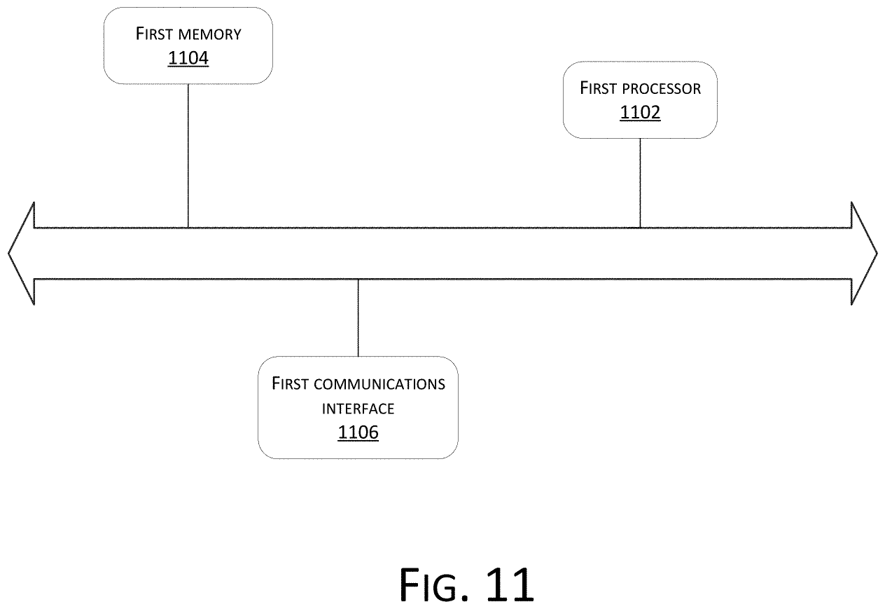

In an example embodiment, a structure of the genetic testing apparatus shown in FIG. 10 are implemented as an electronic device, and the electronic device may be one of various types of devices, such as an all-in-one machine for genetic testing, a server, or the like. As shown in FIG. 11, the electronic device may include a first processor 1102 and a first memory 1104. The first memory 1104 is an example of computer-readable media. For example, the first memory 1104 stores the first obtaining module 1002, the first extraction module 1004, the first processing module 1006, and the first testing module 1008. For another example, the first memory 1104 is configured to store a program for the corresponding electronic device to execute the genetic testing methods in the embodiments shown in FIG. 1 to FIG. 4, FIG. 6, and FIG. 7. The first processor 1102 is configured to execute the program stored in the first memory 1104.

The program includes one or more computer instructions. When the one or more computer instructions are executed by the first processor 1102, the following steps are implemented:

obtaining a to-be-processed genetic sequence, where an average number of gene fragments corresponding to each position in the genetic sequence is less than or equal to a preset threshold;

performing signature extraction on the genetic sequence, and obtaining a gene signature;

enhancing the gene signature, and obtaining an enhanced signature corresponding to the gene signature; and

testing the genetic sequence based on the enhanced signature, and obtaining a testing result.

Further, the first processor 1102 is further configured to execute all or a part of the steps in the embodiments shown in FIG. 1 to FIG. 4, FIG. 6, and FIG. 7.

A structure of the electronic device may further include a first communications interface 1106 for the electronic device to communicate with another device or with a communications network.

In addition, an embodiment of the present disclosure provides a computer storage medium, configured to store a computer software instruction used by the electronic device. The computer storage medium includes a program for performing the genetic testing methods in the method embodiments shown in FIG. 1 to FIG. 4, FIG. 6, and FIG. 7.

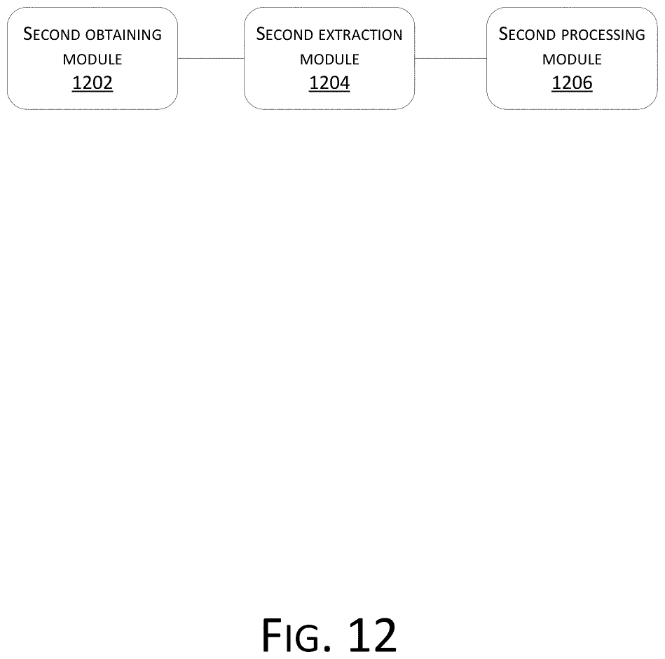

FIG. 12 is a schematic structural diagram of a signature extraction apparatus according to an embodiment of the present disclosure. Referring to FIG. 12, this embodiment provides a signature extraction apparatus, the signature extraction apparatus may perform the foregoing signature extraction method shown in FIG. 5, and the signature extraction apparatus may include: a second obtaining module 1202, a second extraction module 1204, and a second processing module 1206. For example, the second obtaining module 1202 is configured to obtain a to-be-processed genetic sequence, where an average number of gene fragments corresponding to each position in the genetic sequence is less than or equal to a preset threshold;

the second extraction module 1204 is configured to perform signature extraction on the genetic sequence, and obtain a gene signature; and

the second processing module 1206 is configured to enhance the gene signature, and obtain an enhanced signature corresponding to the gene signature, where a quantity of information included in the enhanced signature is greater than a quantity of information included in the gene signature.

The apparatus shown in FIG. 12 may perform the methods in the embodiments shown in FIG. 5 to FIG. 7. For content not described in detail in this embodiment, reference may be made to the related descriptions of the embodiments shown in FIG. 5 to FIG. 7. For an execution process and a technical effect of the technical solution, refer to the descriptions in the embodiments shown in FIG. 5 to FIG. 7. Details are not described herein again.

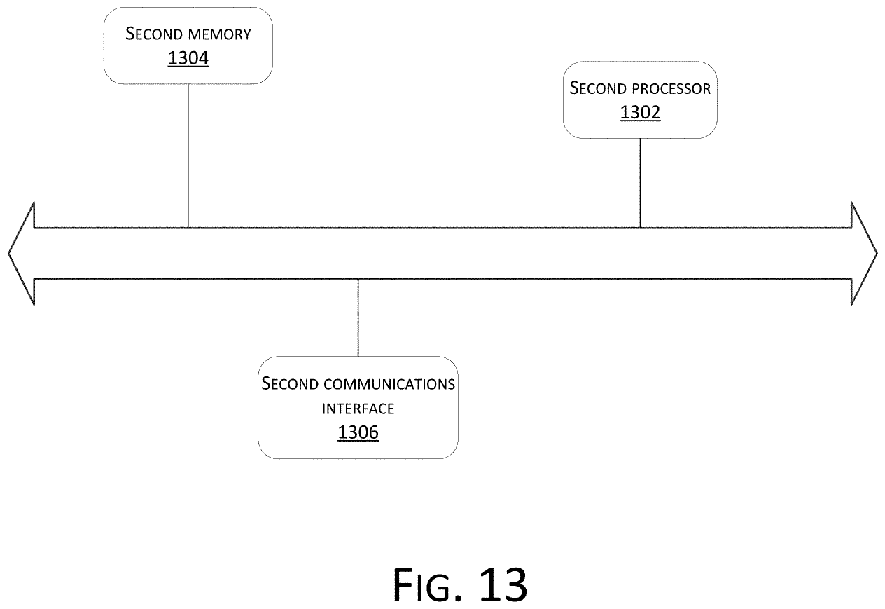

In an example embodiment, a structure of the signature extraction apparatus shown in FIG. 12 are implemented as an electronic device, and the electronic device may be one of various types of devices, such as an all-in-one machine for genetic testing, a server, or the like. As shown in FIG. 13, the electronic device may include a second processor 1302 and a second memory 1304. The second memory 1304 is an example of computer-readable media. For example, the second memory 1304 stores the second obtaining module 1202, the second extraction module 1204, and the second processing module 1206. For another example, the second memory 1304 is configured to store a program for the corresponding electronic device to execute the signature extraction method provided in the embodiment shown in FIG. 5. The second processor 1302 is configured to execute the program stored in the second memory 1304.

The program includes one or more computer instructions. When the one or more computer instructions are executed by the second processor 1302, the following steps are implemented:

obtaining a to-be-processed genetic sequence, where an average number of gene fragments corresponding to each position in the genetic sequence is less than or equal to a preset threshold;

performing signature extraction on the genetic sequence, and obtaining a gene signature; and

enhancing the gene signature, and obtaining an enhanced signature corresponding to the gene signature, where a quantity of information included in the enhanced signature is greater than a quantity of information included in the gene signature.

Further, the second processor 1302 is further configured to perform all or a part of the steps in the embodiment shown in FIG. 5.

A structure of the electronic device may further include a second communications interface 1306 for the electronic device to communicate with another device or with a communications network.

In addition, an embodiment of the present disclosure provides a computer storage medium, configured to store a computer software instruction used by the electronic device. The computer storage medium includes a program for performing the signature extraction method in the method embodiment shown in FIG. 5.

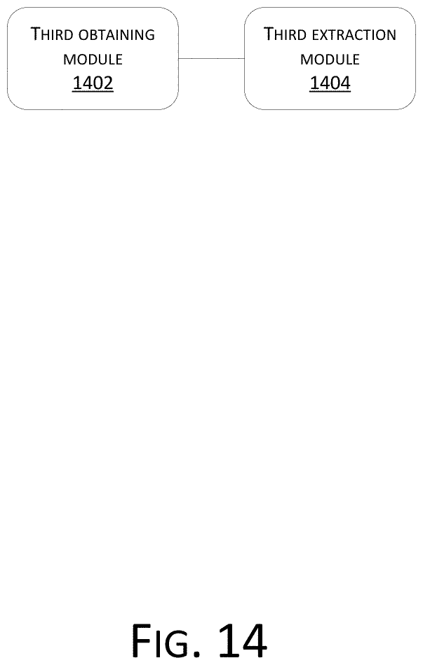

FIG. 14 is a schematic structural diagram of another genetic testing apparatus according to an embodiment of the present disclosure. Referring to FIG. 14, this embodiment provides another genetic testing apparatus, the genetic testing apparatus may perform the genetic testing method shown in FIG. 8, and the genetic testing apparatus may include: a third obtaining module 1402 and a third processing module 1404. For example, the third obtaining module 1402 is configured to determine, in response to a request for invoking genetic testing, a processing resource corresponding to a genetic testing service; and

the third processing module 1404 is configured to perform the following steps by using the processing resource: obtaining a to-be-processed genetic sequence, where an average number of gene fragments corresponding to each position in the genetic sequence is less than or equal to a preset threshold; performing signature extraction on the genetic sequence, and obtaining a gene signature; enhancing the gene signature, and obtaining an enhanced signature corresponding to the gene signature; and testing the genetic sequence based on the enhanced signature, and obtaining a testing result.

The apparatus shown in FIG. 14 may perform the method in the embodiment shown in FIG. 8. For content not described in detail in this embodiment, reference may be made to the related descriptions of the embodiment shown in FIG. 9. For an execution process and a technical effect of the technical solution, refer to the descriptions in the embodiment shown in FIG. 8. Details are not described herein again.

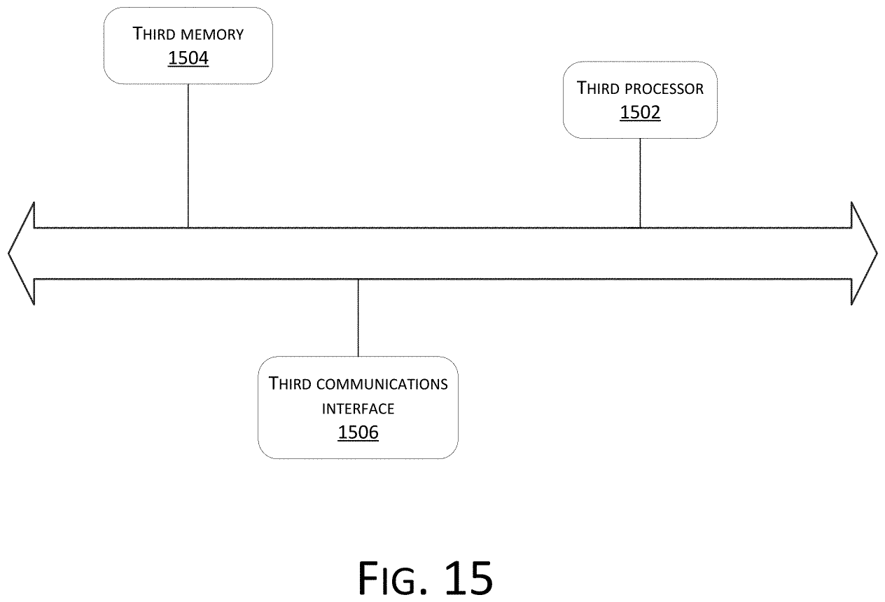

In an example embodiment, a structure of the genetic testing apparatus shown in FIG. 14 are implemented as an electronic device, and the electronic device may be one of various types of devices, such as an all-in-one machine for genetic testing, a server, or the like. As shown in FIG. 15, the electronic device may include a third processor 1502 and a third memory 1504. The third memory 1504 is an example of computer-readable media. For example, the third memory 1504 stores the third obtaining module 1402 and the third extraction module 1404. For another example, the third memory 1504 is configured to store a program for the corresponding electronic device to execute the genetic testing method provided in the embodiment shown in FIG. 8. The third processor 1502 is configured to execute the program stored in the third memory 1504.

The program includes one or more computer instructions. When the one or more computer instructions are executed by the third processor 1502, the following steps are implemented:

determining, in response to a request for invoking genetic testing, a processing resource corresponding to a genetic testing service; and

performing the following steps by using the processing resource: obtaining a to-be-processed genetic sequence, where an average number of gene fragments corresponding to each position in the genetic sequence is less than or equal to a preset threshold; performing signature extraction on the genetic sequence, and obtaining a gene signature; enhancing the gene signature, and obtaining an enhanced signature corresponding to the gene signature; and testing the genetic sequence based on the enhanced signature, and obtaining a testing result.

Further, the third processor 1502 is further configured to perform all or a part of the steps in the embodiment shown in FIG. 8.

A structure of the electronic device may further include a third communications interface 1506 for the electronic device to communicate with another device or with a communications network.

In addition, an embodiment of the present disclosure provides a computer storage medium, configured to store a computer software instruction used by the electronic device. The computer storage medium includes a program for performing the genetic testing methods in the method embodiment shown in FIG. 8.

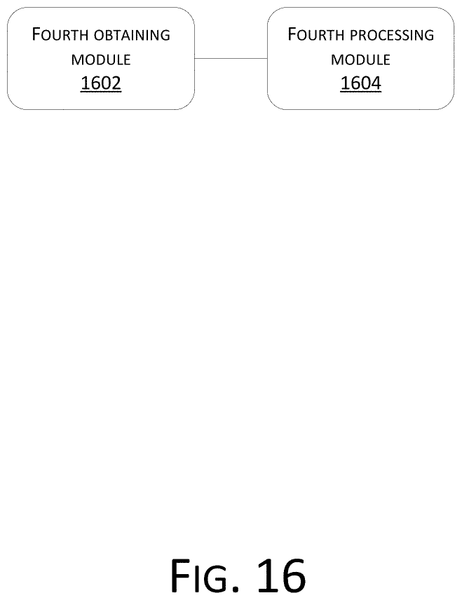

FIG. 16 is a schematic structural diagram of another signature extraction apparatus according to an embodiment of the present disclosure. Referring to FIG. 16, this embodiment provides another signature extraction apparatus, the signature extraction apparatus may perform the signature extraction method shown in FIG. 9, and the signature extraction apparatus may include: a fourth obtaining module 1602 and a fourth processing module 1604.

For example, the fourth obtaining module 1602 is configured to determine, in response to a request for invoking signature extraction, a processing resource corresponding to a signature extraction service; and

the fourth processing module 1604 is configured to perform the following steps by using the processing resource: obtaining a to-be-processed genetic sequence, where an average number of gene fragments corresponding to each position in the genetic sequence is less than or equal to a preset threshold; performing signature extraction on the genetic sequence, and obtaining a gene signature; and enhancing the gene signature, and obtaining an enhanced signature corresponding to the gene signature, where a quantity of information included in the enhanced signature is greater than a quantity of information included in the gene signature.

The apparatus shown in FIG. 16 may perform the method in the embodiment shown in FIG. 9. For content not described in detail in this embodiment, reference may be made to the related descriptions of the embodiment shown in FIG. 9. For an execution process and a technical effect of the technical solution, refer to the descriptions in the embodiment shown in FIG. 10. Details are not described herein again.

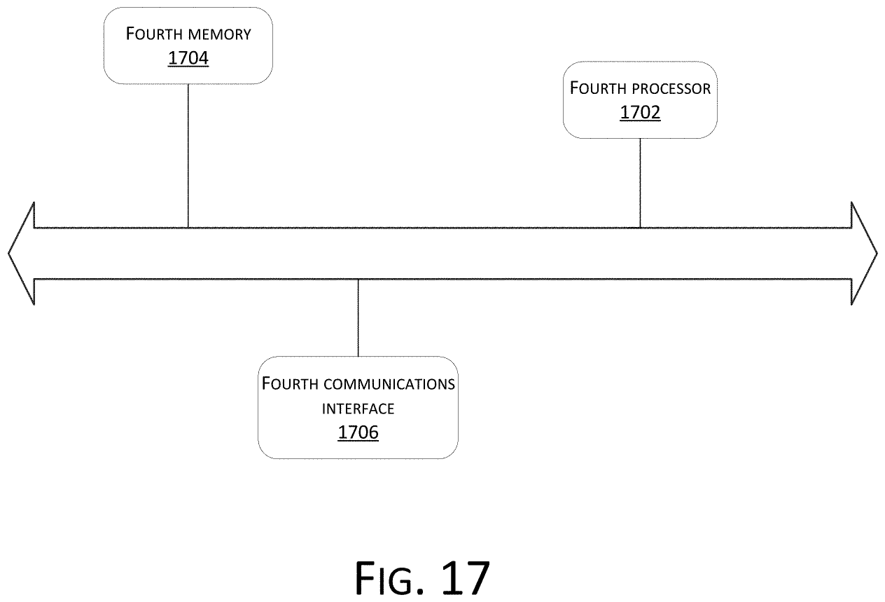

In an example embodiment, a structure of the signature extraction apparatus shown in FIG. 16 are implemented as an electronic device, and the electronic device may be one of various types of devices, such as an all-in-one machine for genetic testing, a server, or the like. As shown in FIG. 17, the electronic device may include a fourth processor 1702 and a fourth memory 1704. The fourth memory 1704 is an example of computer-readable media. For example, the fourth memory 1704 stores the fourth obtaining module 1602 and the fourth processing module 1604. For another example, the fourth memory 1704 is configured to store a program for the corresponding electronic device to execute the signature extraction provided in the embodiment shown in FIG. 10. The fourth processor 1702 is configured to execute the program stored in the fourth memory 1704.

The program includes one or more computer instructions. When the one or more computer instructions are executed by the fourth processor 1702, the following steps are implemented:

determining, in response to a request for invoking signature extraction, a processing resource corresponding to a signature extraction service; and

performing the following steps by using the processing resource: obtaining a to-be-processed genetic sequence, where an average number of gene fragments corresponding to each position in the genetic sequence is less than or equal to a preset threshold; performing signature extraction on the genetic sequence, and obtaining a gene signature; and enhancing the gene signature, and obtaining an enhanced signature corresponding to the gene signature, where a quantity of information included in the enhanced signature is greater than a quantity of information included in the gene signature.

Further, the fourth processor 1702 is further configured to perform all or a part of the steps in the embodiment shown in FIG. 9.

A structure of the electronic device may further include a fourth communications interface 1706 for the electronic device to communicate with another device or with a communications network.

In addition, an embodiment of the present disclosure provides a computer storage medium, configured to store a computer software instruction used by an electronic device. The computer storage medium includes a program for performing the signature extraction method in the method embodiment shown in FIG. 9.

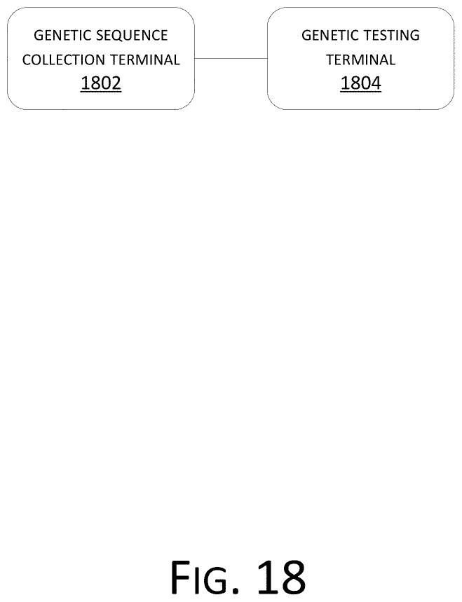

FIG. 18 is a schematic structural diagram of a genetic testing system according to an embodiment of the present disclosure. Referring to FIG. 18, this embodiment provides a genetic testing system, and the genetic testing system may include:

a genetic sequence collection terminal 1802, configured to obtain a to-be-processed genetic sequence, and transmit the genetic sequence to a genetic testing terminal, where an average number of gene fragments corresponding to each position in the genetic sequence is less than or equal to a preset threshold; and

the genetic testing terminal 1804, in a communication connection with the genetic sequence collection terminal 1802, and configured to obtain the to-be-processed genetic sequence; perform signature extraction on the genetic sequence, and obtain a gene signature; enhance the gene signature, and obtain an enhanced signature corresponding to the gene signature; and test the genetic sequence based on the enhanced signature, and obtain a testing result.

The system shown in FIG. 18 may execute the methods in the embodiments shown in FIG. 1 to FIG. 4, FIG. 6, and FIG. 7. For content not described in detail in this embodiment, reference may be made to the related descriptions of the embodiments shown in FIG. 1 to FIG. 4, FIG. 6, and FIG. 7. For an execution process and a technical effect of the technical solution, refer to the descriptions in the embodiments shown in FIG. 1 to FIG. 4, FIG. 6, and FIG. 7. Details are not described herein again.

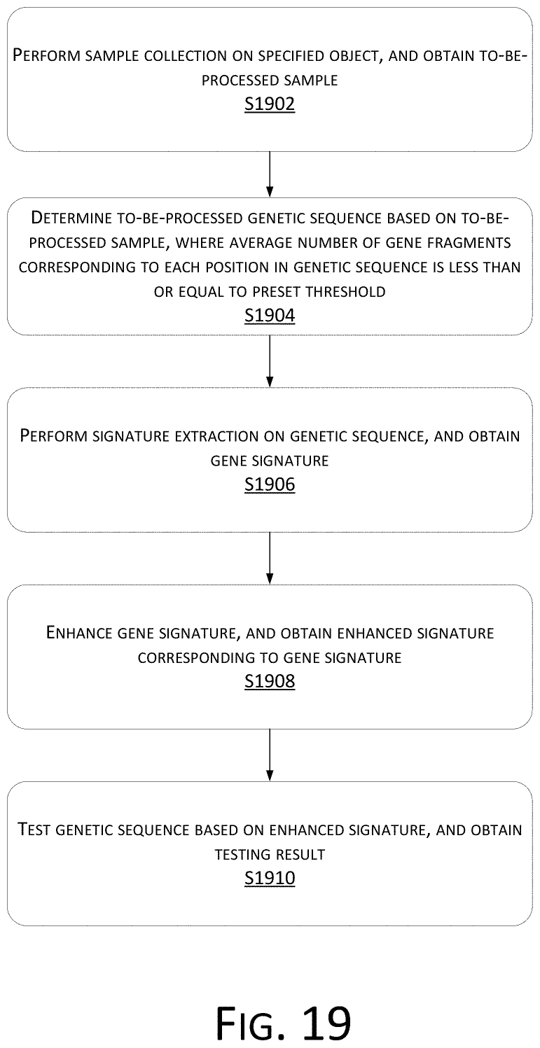

FIG. 19 is a schematic flowchart of another genetic testing method according to an embodiment of the present disclosure. Referring to FIG. 19, this embodiment provides a genetic testing method, and the genetic testing method may be executed by a genetic testing apparatus. The genetic testing apparatus may be implemented as software or a combination of software and hardware. For example, the genetic testing method may include the following steps:

Step S1902: Perform sample collection on a specified object, and obtain a to-be-processed sample.

Step S1904: Determine a to-be-processed genetic sequence based on the to-be-processed sample, where an average number of gene fragments corresponding to each position in the genetic sequence is less than or equal to a preset threshold.

Step S1906: Perform signature extraction on the genetic sequence, and obtain a gene signature.

Step S1908: Enhance the gene signature, and obtain an enhanced signature corresponding to the gene signature.

Step S1910: Test the genetic sequence based on the enhanced signature, and obtain a testing result.

The specified object may be a human object or an animal object. When a user has a genetic testing requirement of the specified object, sample collection may be performed on the specified object to obtain a to-be-processed sample. For example, a gene collection module is disposed on the genetic testing apparatus. Sample collection may be performed on the specified object by using the gene collection module, so that the to-be-processed sample may be obtained. In different application scenarios, the gene collection module may correspond to different structural features. For example, when the to-be-processed sample is a blood sample, the gene collection module may be a blood collector. For example, the blood collector collects blood from a body of the specified object (a person, an animal, or the like), and extracts a to-be-processed genetic sequence based on the extracted blood sample. Similarly, when the to-be-processed sample is a saliva sample, the gene collection module may be a saliva collector. For example, the saliva collector collects saliva from a body of the specified object (a person, an animal, or the like) and extracts a to-be-processed genetic sequence based on the saliva. Similarly, when the to-be-processed sample is a skin sample, the gene collection module may be a skin collector. For example, the skin collector collects skin from a body of the specified object (a person, an animal, or the like) and extracts a to-be-processed genetic sequence based on the skin.

Apparently, a person skilled in the art may also perform sample collection on the specified object in another manner, and obtain the to-be-processed sample, as long as the accuracy and reliability of obtaining the to-be-processed sample can be ensured. Details are not described herein.

After the to-be-processed sample is obtained, the to-be-processed sample may be analyzed and processed to determine the to-be-processed genetic sequence. The average number of gene fragments corresponding to each position in the genetic sequence is less than or equal to the preset threshold. After the genetic sequence is obtained, signature extraction may be performed on the genetic sequence, and the gene signature is obtained; then the gene signature is enhanced, an enhanced signature corresponding to the gene signature is obtained, and the genetic sequence may be tested based on the enhanced signature to obtain the testing result.

It should be noted that a specific implementation manner, a specific implementation principle, and a specific implementation effect of step S1904 to step S1910 in this embodiment are similar to the specific implementation manner, the specific implementation principle, and the specific implementation effect of step S202 to step S208 in the embodiment corresponding to FIG. 2. Reference may be made to the foregoing statements, and details are not described herein again. In addition, the method in this embodiment may further include the methods of the embodiments shown in FIG. 2 to FIG. 4, FIG. 6, and FIG. 7. For content not described in detail in this embodiment, reference may be made to the related descriptions of the embodiments shown in FIG. 2 to FIG. 4, FIG. 6, and FIG. 7. For an execution process and a technical effect of the technical solution, refer to the descriptions in the embodiments shown in FIG. 2 to FIG. 4, FIG. 6, and FIG. 7. Details are not described herein again.

In the genetic testing method provided by this embodiment, sample collection is performed on the specified object, and the to-be-processed sample is obtained; the to-be-processed genetic sequence is determined based on the to-be-processed sample; signature extraction is performed on the genetic sequence, and the gene signature is obtained; the gene signature is enhanced, and the enhanced signature corresponding to the gene signature is obtained; furthermore, the genetic sequence may be tested based on the enhanced signature obtained, and the testing result is obtained. In this way, not only may the specified object participate in the entire genetic testing and is genetic testing precision ensured, but also data processing costs and a quantity of processed data are further effectively reduced, thereby effectively achieving relatively precise testing based on low-depth gene data, further improving practicality of the method, and facilitating promotion and application in the market.

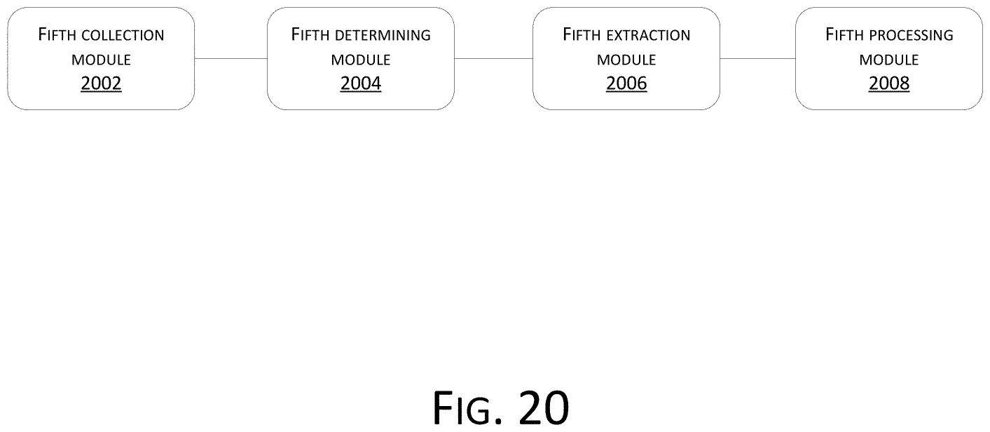

FIG. 20 is a schematic structural diagram of still another genetic testing apparatus according to an embodiment of the present disclosure. Referring to FIG. 20, this embodiment provides still another genetic testing apparatus, the genetic testing apparatus may perform the genetic testing method shown in FIG. 19. For example, the genetic testing apparatus may include: a fifth collection module 2002, a fifth determining module 2004, a fifth extraction module 2006, and a fifth processing module 2008.

The fifth collection module 2002 is configured to perform sample collection on a specified object and obtain a to-be-processed sample.

The fifth determination module 2004 is configured to determine a to-be-processed genetic sequence based on the to-be-processed sample, where an average number of gene fragments corresponding to each position in the genetic sequence is less than or equal to a preset threshold.

The fifth extraction module 2006 is configured to perform signature extraction on the genetic sequence and obtain a gene signature.

The fifth processing module 2008 is configured to enhance the gene signature and obtain an enhanced signature corresponding to the gene signature.

The fifth processing module 2008 is further configured to test the genetic sequence based on the enhanced signature and obtain a testing result.

The genetic testing apparatus in this embodiment may perform the method in the embodiment shown in FIG. 19. For content not described in detail in this embodiment, reference may be made to the related descriptions of the embodiment shown in FIG. 19. For an execution process and a technical effect of the technical solution, refer to the descriptions in the embodiment shown in FIG. 19. Details are not described herein again.

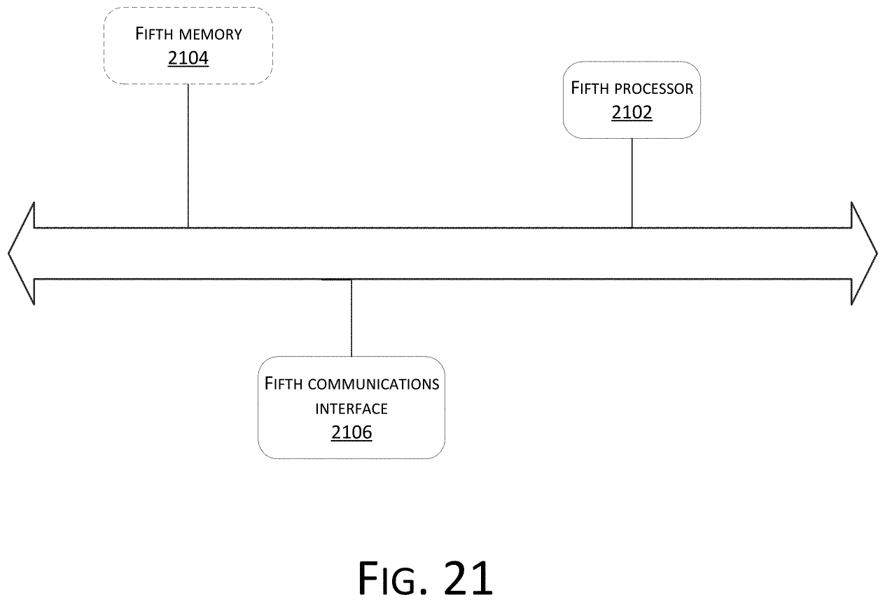

In an example embodiment, a structure of the genetic testing apparatus shown in FIG. 20 are implemented as an electronic device, and the electronic device may be one of various types of devices, such as an all-in-one machine for genetic testing, a server, or the like. As shown in FIG. 21, the electronic device may include a fifth processor 2102 and a fifth memory 2104. The fifth memory 2104 is an example of computer-readable media. For example, the fifth memory 2104 stores the fifth collection module 2002, the fifth determining module 2004, the fifth extraction module 2006, and the fifth processing module 2008. For another example,

the fifth memory 2104 is configured to store a program for the corresponding electronic device to execute the genetic testing method provided in the embodiment shown in FIG. 19. The fifth processor 2102 is configured to execute the program stored in the fifth memory 2104.

The program includes one or more computer instructions. When the one or more computer instructions are executed by the fifth processor 2102, the following steps are implemented:

performing sample collection on a specified object, and obtaining a to-be-processed sample;

determining a to-be-processed genetic sequence based on the to-be-processed sample, where an average number of gene fragments corresponding to each position in the genetic sequence is less than or equal to a preset threshold;

performing signature extraction on the genetic sequence, and obtaining a gene signature;

enhancing the gene signature, and obtaining an enhanced signature corresponding to the gene signature; and

testing the genetic sequence based on the enhanced signature, and obtaining a testing result.

Further, the fifth processor 2102 is further configured to perform all or a part of the steps in the embodiment shown in FIG. 19.

A structure of the electronic device may further include a fifth communications interface 2106 for the electronic device to communicate with another device or with a communications network.

In addition, an embodiment of the present disclosure provides a computer storage medium, configured to store a computer software instruction used by an electronic device. The computer storage medium includes a program for performing the genetic testing method in the method embodiment shown in FIG. 19.

The apparatus embodiments described above are only examples. The units described as separate components may or may not be physically separated, and the components displayed as units may or may not be physical units. That is, the units may be located in one place, or may be distributed on a plurality of network units. Some or all of the modules may be selected according to actual needs to achieve the objectives of the solutions of the embodiments. Those of ordinary skill in the art may understand and implement the embodiments without creative efforts.

Through the description of the above implementations, a person skilled in the art may clearly understand that each implementation may be realized by using a necessary general hardware platform, and may certainly be implemented by a combination of hardware and software. Based on such an understanding, the part of the above technical solutions, which is essential or contributes to the prior art, may be embodied in the form of a computer product. The present disclosure may take the form of a computer program product which is embodied on one or more computer-usable storage media (including, but not limited to, a disk storage, a CD-ROM, an optical storage, and the like) having computer-usable program code contained therein.

The present disclosure is described with reference to the flowcharts and/or block diagrams of the method, the device (system), and the computer program product according to the embodiments of the present disclosure. It should be understood that computer program instructions may be used to implement each process and/or each block in the flowcharts and/or the block diagrams and a combination of a process and/or a block in the flowcharts and/or the block diagrams. The computer program instructions may be provided for a general-purpose computer, a dedicated computer, an embedded processor, or a processor of another programmable device to generate a machine, so that the instructions executed by a computer or a processor of another programmable generate an apparatus for implementing a specific function in one or more processes in the flowcharts and/or in one or more blocks in the block diagrams.

The computer program instructions may be stored in a computer readable memory that can instruct the computer or another programmable device to work in a specific manner, so that the instructions stored in the computer readable memory generate an artifact that includes an instruction apparatus. The instruction apparatus implements a specific function in one or more processes in the flowcharts and/or in one or more blocks in the block diagrams.

The computer program instructions may also be loaded onto a computer or another programmable device, so that a series of operation steps are performed on the computer or another programmable device to generate computer-implemented processing. Therefore, the instructions executed on the computer or another programmable device are used to provide steps for implementing a specific function in one or more processes in the flowcharts and/or in one or more blocks in the block diagrams.

In a typical configuration, a computing device includes one or more processors (CPU), an input/output interface, a network interface, and a memory.

The memory may include a volatile memory on a computer-readable medium, a random-access memory (RAM), and/or a non-volatile memory, and the like, such as a read-only memory (ROM) or a flash random access memory (flash RAM). The memory is an example of the computer-readable media.

Computer-readable media include nonvolatile and volatile, removable and non-removable media employing any method or technique to achieve information storage. The information may be computer readable instructions, data structures, modules of programs, or other data. Examples of computer storage media include, but are not limited to, a phase-change random access memory (PRAM), a static random access memory (SRAM), a dynamic random access memory (DRAM), other types of random access memories (RAM), a read-only memory (ROM), an electrically erasable programmable read-only memory (EEPROM), a flash memory or other memory technologies, a compact disc read-only memory (CD-ROM), a digital versatile disc (DVD) or other optical memories, a magnetic cassette tape, a magnetic tape, a magnetic disk storage or other magnetic storage devices or any other non-transmission medium, which may be used to store information that can be accessed by a computing device. As defined herein, the computer-readable media do not include transitory media, such as modulated data signals and carriers.

Finally, it should be noted that the above embodiments are merely used for illustrating, rather than limiting, the technical solutions of the present disclosure. Although the present disclosure is described in detail with reference to the foregoing embodiments, it should be understood by those of ordinary skill in the art that modifications may still be made to the technical solutions described in the foregoing embodiments, or equivalent substitutions may be applied to part of the technical signatures therein; and the modifications or substitutions do not cause the essence of corresponding technical solutions to depart from the spirit and scope of the technical solutions in the embodiments of the present disclosure.

The present disclosure may further be understood with clauses as follows:

Clause 1. A genetic testing method, comprising:

obtaining a to-be-processed genetic sequence, wherein an average number of gene fragments corresponding to each position in the genetic sequence is less than or equal to a preset threshold;

performing signature extraction on the genetic sequence, and obtaining a gene signature;

enhancing the gene signature, and obtaining an enhanced signature corresponding to the gene signature; and

testing the genetic sequence based on the enhanced signature, and obtaining a testing result.

Clause 2. The method according to clause 1, wherein the step of performing signature extraction on the genetic sequence and obtaining a gene signature comprises:

determining a to-be-analyzed gene fragment corresponding to the genetic sequence; and

performing signature extraction on the to-be-analyzed gene fragment, and obtaining the gene signature.

Clause 3. The method according to clause 2, wherein the step of determining a to-be-analyzed gene fragment corresponding to the genetic sequence comprises:

obtaining reference data and a plurality of initial gene fragments comprised in the genetic sequence; and

performing matching between the reference data and the genetic sequence to determine the to-be-analyzed gene fragment among the plurality of initial gene fragments, wherein there is a base in the to-be-analyzed gene fragment and that does not match the reference data, and a proportion of the unmatched base in the to-be-analyzed gene fragment is greater than a preset base threshold.

Clause 4. The method according to clause 2, wherein the step of performing signature extraction on the to-be-analyzed gene fragment and obtaining the gene signature comprises:

obtaining a base quality comprised in the to-be-analyzed gene fragment;

determining, based on the base quality, a confidence level corresponding to the to-be-analyzed gene fragment; and

performing signature extraction on the to-be-analyzed gene fragment based on the confidence level corresponding to the to-be-analyzed gene fragment, and obtaining the gene signature.

Clause 5. The method according to clause 1, wherein the step of enhancing the gene signature and obtaining an enhanced signature corresponding to the gene signature comprises:

obtaining a convolutional neural network model used for enhancing the gene signature; and

enhancing the gene signature based on the convolutional neural network model, and obtaining the enhanced signature corresponding to the gene signature.

Clause 6. The method according to any one of clauses 1 to 5, wherein a quantity of information comprised in the enhanced signature is greater than a quantity of information comprised in the gene signature.

Clause 7. The method according to any one of clauses 1 to 5, wherein a data magnitude of the enhanced signature is the same as a data magnitude of the gene signature.

Clause 8. The method according to any one of clauses 1 to 5, wherein the step of testing the genetic sequence based on the enhanced signature, and obtaining a testing result comprises:

obtaining, based on the enhanced signature, mutation reference information corresponding to the enhanced signature, wherein the mutation reference information comprises at least one of the following: prediction information of 21 genotypes, zygote prediction information, first allele mutation length information, and second allele mutation length information; and

obtaining a mutation testing result based on the mutation reference information.

Clause 9. The method according to any one of clauses 1 to 5, wherein the step of testing the genetic sequence based on the enhanced signature, and obtaining a testing result comprises:

inputting the enhanced signature into a three-dimensional network model, and obtaining the testing result, wherein the three-dimensional network model is trained to test a genetic sequence based on a gene signature.

Clause 10. A signature extraction method, comprising:

obtaining a to-be-processed genetic sequence, wherein an average number of gene fragments corresponding to each position in the genetic sequence is less than or equal to a preset threshold;

performing signature extraction on the genetic sequence, and obtaining a gene signature;

enhancing the gene signature, and obtaining an enhanced signature corresponding to the gene signature, wherein a quantity of information comprised in the enhanced signature is greater than a quantity of information comprised in the gene signature.

Clause 11. A genetic testing method, comprising:

determining, in response to a request for invoking genetic testing, a processing resource corresponding to a genetic testing service; and

performing the following steps by using the processing resource: obtaining a to-be-processed genetic sequence, wherein an average number of gene fragments corresponding to each position in the genetic sequence is less than or equal to a preset threshold; performing signature extraction on the genetic sequence, and obtaining a gene signature; enhancing the gene signature, and obtaining an enhanced signature corresponding to the gene signature; and testing the genetic sequence based on the enhanced signature, and obtaining a testing result.

Clause 12. A signature extraction method, comprising:

determining, in response to a request for invoking signature extraction, a processing resource corresponding to a signature extraction service; and

performing the following steps by using the processing resource: obtaining a to-be-processed genetic sequence, wherein an average number of gene fragments corresponding to each position in the genetic sequence is less than or equal to a preset threshold; performing signature extraction on the genetic sequence, and obtaining a gene signature; and enhancing the gene signature, and obtaining an enhanced signature corresponding to the gene signature, wherein a quantity of information comprised in the enhanced signature is greater than a quantity of information comprised in the gene signature.

Clause 13. A genetic testing method, comprising:

performing sample collection on a specified object, and obtaining a to-be-processed sample;

determining a to-be-processed genetic sequence based on the to-be-processed sample, wherein an average number of gene fragments corresponding to each position in the genetic sequence is less than or equal to a preset threshold;

performing signature extraction on the genetic sequence, and obtaining a gene signature;

enhancing the gene signature, and obtaining an enhanced signature corresponding to the gene signature; and

testing the genetic sequence based on the enhanced signature, and obtaining a testing result.

Clause 14. A genetic testing system, comprising:

a genetic sequence collection terminal, configured to obtain a to-be-processed genetic sequence, and transmit the genetic sequence to a genetic testing terminal, wherein an average number of gene fragments corresponding to each position in the genetic sequence is less than or equal to a preset threshold; and

the genetic testing terminal, in a communication connection with the genetic sequence collection terminal, and configured to obtain the to-be-processed genetic sequence; perform signature extraction on the genetic sequence and obtain a gene signature; enhance the gene signature and obtain an enhanced signature corresponding to the gene signature; and test the genetic sequence based on the enhanced signature and obtain a testing result.

What is claimed is:

1. A method comprising:
obtaining a genetic sequence, an average number of gene fragments corresponding to a position in the genetic sequence being less than or equal to a preset threshold;
performing signature extraction on the genetic sequence to obtain a gene signature;
enhancing the gene signature to obtain an enhanced signature corresponding to the gene signature; and
testing the genetic sequence based on the enhanced signature.

2. The method according to claim 1, further comprising obtaining a testing result.

3. The method according to claim 1, wherein the performing signature extraction on the genetic sequence to obtain the gene signature comprises:
determining a to-be-analyzed gene fragment corresponding to the genetic sequence;
performing signature extraction on the to-be-analyzed gene fragment; and
obtaining the gene signature.

4. The method according to claim 3, wherein the determining the to-be-analyzed gene fragment corresponding to the genetic sequence comprises:
obtaining reference data and a plurality of initial gene fragments included in the genetic sequence; and
performing matching between the reference data and the genetic sequence to determine the to-be-analyzed gene fragment among the plurality of initial gene fragments.

5. The method according to claim 4, wherein:
there is a base in the to-be-analyzed gene fragment and that does not match the reference data; and
a proportion of the base in the to-be-analyzed gene fragment is greater than a preset base threshold.

6. The method according to claim 3, wherein the performing signature extraction on the to-be-analyzed gene fragment comprises:
obtaining a base quality included in the to-be-analyzed gene fragment;
determining, based on the base quality, a confidence level corresponding to the to-be-analyzed gene fragment; and
performing signature extraction on the to-be-analyzed gene fragment based on the confidence level corresponding to the to-be-analyzed gene fragment.

7. The method according to claim 1, wherein the enhancing the gene signature to obtain the enhanced signature corresponding to the gene signature comprises:
obtaining a convolutional neural network model used for enhancing the gene signature;
enhancing the gene signature based on the convolutional neural network model; and
obtaining the enhanced signature corresponding to the gene signature.

8. The method according to claim 1, wherein a quantity of information included in the enhanced signature is greater than a quantity of information included in the gene signature.

9. The method according to claim 1, wherein a data magnitude of the enhanced signature is the same as a data magnitude of the gene signature.

10. The method according to claim 1, wherein the testing the genetic sequence based on the enhanced signature comprises:
obtaining, based on the enhanced signature, mutation reference information corresponding to the enhanced signature, wherein the mutation reference information comprises at least one of the following:
prediction information of 21 genotypes;
zygote prediction information;
first allele mutation length information; and
second allele mutation length information.

11. The method according to claim 10, wherein the testing the genetic sequence based on the enhanced signature comprises obtaining a mutation testing result based on the mutation reference information.

12. The method according to claim 1, wherein the testing the genetic sequence based on the enhanced signature comprises:
inputting the enhanced signature into a three-dimensional network model, wherein the three-dimensional network model is trained to test the genetic sequence based on the gene signature.

13. A device comprising:
one or more processors; and
one or more memories storing thereon computer-readable instructions that, when executed by the one or more processors, cause the one or more processors to perform acts comprising:
obtaining a genetic sequence, an average number of gene fragments corresponding to a position in the genetic sequence being less than or equal to a preset threshold;
performing signature extraction on the genetic sequence to obtaining a gene signature;
enhancing the gene signature to obtain an enhanced signature corresponding to the gene signature, a quantity of information included in the enhanced signature being greater than a quantity of information included in the gene signature.

14. The device according to claim 13, wherein the performing signature extraction on the genetic sequence to obtain the gene signature comprises:
determining a to-be-analyzed gene fragment corresponding to the genetic sequence;
performing signature extraction on the to-be-analyzed gene fragment; and
obtaining the gene signature.

15. The device according to claim 14, wherein the determining the to-be-analyzed gene fragment corresponding to the genetic sequence comprises:
obtaining reference data and a plurality of initial gene fragments included in the genetic sequence; and
performing matching between the reference data and the genetic sequence to determine the to-be-analyzed gene fragment among the plurality of initial gene fragments.

16. The device according to claim 15, wherein:
there is a base in the to-be-analyzed gene fragment and that does not match the reference data; and
a proportion of the base in the to-be-analyzed gene fragment is greater than a preset base threshold.

17. The device according to claim 14, wherein the performing signature extraction on the to-be-analyzed gene fragment comprises:
obtaining a base quality included in the to-be-analyzed gene fragment;
determining, based on the base quality, a confidence level corresponding to the to-be-analyzed gene fragment; and
performing signature extraction on the to-be-analyzed gene fragment based on the confidence level corresponding to the to-be-analyzed gene fragment.

18. The device according to claim 13, wherein the enhancing the gene signature to obtain the enhanced signature corresponding to the gene signature comprises:
obtaining a convolutional neural network model used for enhancing the gene signature;
enhancing the gene signature based on the convolutional neural network model; and
obtaining the enhanced signature corresponding to the gene signature.

19. The device according to claim 13, further comprising:
obtaining, based on the enhanced signature, mutation reference information corresponding to the enhanced signature, wherein the mutation reference information comprises at least one of the following:
prediction information of 21 genotypes;
zygote prediction information;
first allele mutation length information; and
second allele mutation length information.

20. One or more memories storing thereon computer-readable instructions that, when executed by one or more processors, cause the one or more processors to perform acts comprising:
performing sample collection on a specified object to obtain a to-be-processed sample;
determining a genetic sequence based on the to-be-processed sample, an average number of gene fragments corresponding to each position in the genetic sequence being less than or equal to a preset threshold;
performing signature extraction on the genetic sequence to obtain a gene signature;
enhancing the gene signature to obtain an enhanced signature corresponding to the gene signature; and
testing the genetic sequence based on the enhanced signature to obtain a testing result.
